SEQUENCE LISTING (16 sequences)

SEQ ID NO: 1 - protein, 338 aa, Erwinia chrysanthemi
MQITIKAHIGGDLGVSGLGAQGLKGLNSAASSLGSSVDKLSSTIDKLTSALTSMMFGGALAQGLGASSKGLGMSNQLGQSFGNGAQGASNLLSVPKSGGDALSKMFDKALDDLLGHDTVTKLTNQSNQLANSMLNASQMTQGNMNAFGSGVNNALSSILGNGLGQSMSGFSQPSLGAGGLQGLSGAGAFNQLGNAIGMGVGQNAALSALSNVSTHVDGNNRHFVDKEDRGMAKEIGQFMDQYPEIFGKPEYQKDGWSSPKTDDKSWAKALSKPDDDGMTGASMDKFRQAMGMIKSAVAGDTGNTNLNLRGAGGASLGIDAAVVGDKIANMSLGKLANA

SEQ ID NO: 2 - DNA, 2141 bp, Erwinia chrysanthemi
cgattttacccgggtgaacgtgctatgaccgacagcatcacggtattcgacaccgttacggcgtttatggccgcgatgaaccggcatcaggcggcgcgctggtcgccgcaatccggcgtcgatctggtatttcagtttggggacaccgggcgtgaactcatgatgcagattcagccggggcagcaatatcccggcatgttgcgcacgctgctcgctcgtcgttatcagcaggcggcagagtgcgatggctgccatctgtgcctgaacggcagcgatgtattgatcctctggtggccgctgccgtcggatcccggcagttatccgcaggtgatcgaacgtttgtttgaactggcgggaatgacgttgccgtcgctatccatagcaccgacggcgcgtccgcagacagggaacggacgcgcccgatcattaagataaaggcggctttttttattgcaaaacggtaacggtgaggaaccgtttcaccgtcggcgtcactcagtaacaagtatccatcatgatgcctacatcgggatcggcgtgggcatccgttgcagatacttttgcgaacacctgacatgaatgaggaaacgaaattatgcaaattacgatcaaagcgcacatcggcggtgatttgggcgtctccggtctggggctgggtgctcagggactgaaaggactgaattccgcggcttcatcgctgggttccagcgtggataaactgagcagcaccatcgataagttgacctccgcgctgacttcgatgatgtttggcggcgcgctggcgcaggggctgggcgccagctcgaaggggctggggatgagcaatcaactgggccagtctttcggcaatggcgcgcagggtgcgagcaacctgctatccgtaccgaaatccggcggcgatgcgttgtcaaaaatgtttgataaagcgctggacgatctgctgggtcatgacaccgtgaccaagctgactaaccagagcaaccaactggctaattcaatgctgaacgccagccagatgacccagggtaatatgaatgcgttcggcagcggtgtgaacaacgcactgtcgtccattctcggcaacggtctcggccagtcgatgagtggcttctctcagccttctctgggggcaggcggcttgcagggcctgagcggcgcgggtgcattcaaccagttgggtaatgccatcggcatgggcgtggggcagaatgctgcgctgagtgcgttgagtaacgtcagcacccacgtagacggtaacaaccgccactttgtagataaagaagatcgcggcatggcgaaagagatcggccagtttatggatcagtatccggaaatattcggtaaaccggaataccagaaagatggctggagttcgccgaagacggacgacaaatcctgggctaaagcgctgagtaaaccggatgatgacggtatgaccggcgccagcatggacaaattccgtcaggcgatgggtatgatcaaaagcgcggtggcgggtgataccggcaataccaacctgaacctgcgtggcgcgggcggtgcatcgctgggtatcgatgcggctgtcgtcggcgataaaatagccaacatgtcgctgggtaagctggccaacgcctgataatctgtgctggcctgataaagcggaaacgaaaaaagagacggggaagcctgtctcttttcttattatgcggtttatgcggttacctggaccggttaatcatcgtcatcgatctggtacaaacgcacattttcccgttcattcgcgtcgttacgcgccacaatcgcgatggcatcttcctcgtcgctcagattgcgcggctgatggggaacgccgggtggaatatagagaaactcgccggccagatggagacacgtctgcgataaatctgtgccgtaacgtgtttctatccgcccctttagcagatagattgcggtttcgtaatcaacatggtaatgcggttccgcctgtgcgccggccgggatcaccacaatattcatagaaagctgtcttgcacctaccgtatcgcgggagataccgacaaaatagggcagtttttgcgtggtatccgtggggtgttccggcctgacaatcttgagttggttcgtcatcatctttctccatctgggcgacctgatcggtt

SEQ ID NO: 3 - protein, 403 aa, Erwinia amylovora
MSLNTSGLGASTMQISIGGAGGNNGLLGTSRQNAGLGGNSALGLGGGNQNDTVNQLAGLLTGMMMMMSMMGGGGLMGGGLGGGLGNGLGGSGGLGEGLSNALNDMLGGSLNTLGSKGGNNTTSTTNSPLDQALGINSTSQNDDSTSGTDSTSDSSDPMQQLLKMFSEIMQSLFGDGQDGTQGSSSGGKQPTEGEQNAYKKGVTDALSGLMGNGLSQLLGNGGLGGGQGGNAGTGLDGSSLGGKGLQNLSGPVDYQQLGNAVGTGIGMKAGIQALNDIGTHRHSSTRSFVNKGDRAMAKEIGQFMDQYPEVFGKPQYQKGPGQEVKTDDKSWAKALSKPDDDGMTPASMEQFNKAKGMIKRPMAGDTGNGNLQARGAGGSSLGIDAMMAGDAINNMALGKLGAA

SEQ ID NO: 4 - DNA, 1288 bp, Erwinia amylovora
aagcttcggcatggcacgtttgaccgttgggtcggcagggtacgtttgaattattcataagaggaatacgttatgagtctgaatacaagtgggctgggagcgtcaacgatgcaaatttctatcggcggtgcgggcggaaataacgggttgctgggtaccagtcgccagaatgctgggttgggtggcaattctgcactggggctgggcggcggtaatcaaaatgataccgtcaatcagctggctggcttactcaccggcatgatgatgatgatgagcatgatgggcggtggtgggctgatgggcggtggcttaggcggtggcttaggtaatggcttgggtggctcaggtggcctgggcgaaggactgtcgaacgcgctgaacgatatgttaggcggttcgctgaacacgctgggctcgaaaggcggcaacaataccacttcaacaacaaattccccgctggaccaggcgctgggtattaactcaacgtcccaaaacgacgattccacctccggcacagattccacctcagactccagcgacccgatgcagcagctgctgaagatgttcagcgagataatgcaaagcctgtttggtgatgggcaagatggcacccagggcagttcctctgggggcaagcagccgaccgaaggcgagcagaacgcctataaaaaaggagtcactgatgcgctgtcgggcctgatgggtaatggtctgagccagctccttggcaacgggggactgggaggtggtcagggcggtaatgctggcacgggtcttgacggttcgtcgctgggcggcaaagggctgcaaaacctgagcgggccggtggactaccagcagttaggtaacgccgtgggtaccggtatcggtatgaaagcgggcattcaggcgctgaatgatatcggtacgcacaggcacagttcaacccgttctttcgtcaataaaggcgatcgggcgatggcgaaggaaatcggtcagttcatggaccagtatcctgaggtgtttggcaagccgcagtaccagaaaggcccgggtcaggaggtgaaaaccgatgacaaatcatgggcaaaagcactgagcaagccagatgacgacggaatgacaccagccagtatggagcagttcaacaaagccaagggcatgatcaaaaggcccatggcgggtgataccggcaacggcaacctgcaggcacgcggtgccggtggttcttcgctgggtattgatgccatgatggccggtgatgccattaacaatatggcacttggcaagctgggcgcggcttaagctt

SEQ ID NO: 5 - protein, 447 aa, Erwinia amylovora
MSILTLNNNTSSSPGLFQSGGDNGLGGHNANSALGQQPIDRQTIEQMAQLLAELLKSLLSPQSGNAATGAGGNDQTTGVGNAGGLNGRKGTAGTTPQSDSQNMLSEMGNNGLDQAITPDGQGGGQIGDNPLLKAMLKLIARMMDGQSDQFGQPGTGNNSASSGTSSSGGSPFNDLSGGKAPSGNSPSGNYSPVSTFSPPSTPTSPTSPLDFPSSPTKAAGGSTPVTDHPDPVGSAGIGAGNSVAFTSAGANQTVLHDTITVKAGQVFDGKGQTFTAGSELGDGGQSENQKPLFILEDGASLKNVTMGDDGADGIHLYGDAKIDNLHVTNVGEDAITVKPNSAGKKSHVEITNSSFEHASDKILQLNADTNLSVDNVKAKDFGTFVRTNGGQQGNWDLNLSHISAEDGKFSFVKSDSEGLNVNTSDISLGDVENHYKVPMSANLKVAE

SEQ ID NO: 6 - DNA, 1344 bp, Erwinia amylovora
atgtcaattcttacgcttaacaacaatacctcgtcctcgccgggtctgttccagtccgggggggacaacgggcttggtggtcataatgcaaattctgcgttggggcaacaacccatcgatcggcaaaccattgagcaaatggctcaattattggcggaactgttaaagtcactgctatcgccacaatcaggtaatgcggcaaccggagccggtggcaatgaccagactacaggagttggtaacgctggcggcctgaacggacgaaaaggcacagcaggaaccactccgcagtctgacagtcagaacatgctgagtgagatgggcaacaacgggctggatcaggccatcacgcccgatggccagggcggcgggcagatcggcgataatcctttactgaaagccatgctgaagcttattgcacgcatgatggacggccaaagcgatcagtttggccaacctggtacgggcaacaacagtgcctcttccggtacttcttcatctggcggttccccttttaacgatctatcaggggggaaggccccttccggcaactccccttccggcaactactctcccgtcagtaccttctcacccccatccacgccaacgtcccctacctcaccgcttgatttcccttcttctcccaccaaagcagccgggggcagcacgccggtaaccgatcatcctgaccctgttggtagcgcgggcatcggggccggaaattcggtggccttcaccagcgccggcgctaatcagacggtgctgcatgacaccattaccgtgaaagcgggtcaggtgtttgatggcaaaggacaaaccttcaccgccggttcagaattaggcgatggcggccagtctgaaaaccagaaaccgctgtttatactggaagacggtgccagcctgaaaaacgtcaccatgggcgacgacggggcggatggtattcatctttacggtgatgccaaaatagacaatctgcacgtcaccaacgtgggtgaggacgcgattaccgttaagccaaacagcgcgggcaaaaaatcccacgttgaaatcactaacagttccttcgagcacgcctctgacaagatcctgcagctgaatgccgatactaacctgagcgttgacaacgtgaaggccaaagactttggtacttttgtacgcactaacggcggtcaacagggtaactgggatctgaatctgagccatatcagcgcagaagacggtaagttctcgttcgttaaaagcgatagcgaggggctaaacgtcaataccagtgatatctcactgggtgatgttgaaaaccactacaaagtgccgatgtccgccaacctgaaggtggctgaatga

SEQ ID NO: 7 - protein, 341 aa, Pseudomonas syringae
MQSLSLNSSSLQTPAMALVLVRPEAETTGSTSSKALQEVVVKLAEELMRNGQLDDSSPLGKLLAKSMAADGKAGGGIEDVIAALDKLIHEKLGDNFGASADSASGTGQQDLMTQVLNGLAKSMLDDLLTKQDGGTSFSEDDMPMLNKIAQFMDDNPAQFPKPDSGSWVNELKEDNFLDGDETAAFRSALDIIGQQLGNQQSDAGSLAGTGGGLGTPSSFSNNSSVMGDPLIDANTGPGDSGNTRGEAGQLIGELIDRGLQSVLAGGGLGTPVNTPQTGTSANGGQSAQDLDQLLGGLLLKGLEATLKDAGQTGTDVQSSAAQIATLLVSTLLQGTRNQAAA

SEQ ID NO: 8 - DNA, 1026 bp, Pseudomonas syringae
atgcagagtctcagtcttaacagcagctcgctgcaaaccccggcaatggcccttgtcctggtacgtcctgaagccgagacgactggcagtacgtcgagcaaggcgcttcaggaagttgtcgtgaagctggccgaggaactgatgcgcaatggtcaactcgacgacagctcgccattgggaaaactgttggccaagtcgatggccgcagatggcaaggcgggcggcggtattgaggatgtcatcgctgcgctggacaagctgatccatgaaaagctcggtgacaacttcggcgcgtctgcggacagcgcctcgggtaccggacagcaggacctgatgactcaggtgctcaatggcctggccaagtcgatgctcgatgatcttctgaccaagcaggatggcgggacaagcttctccgaagacgatatgccgatgctgaacaagatcgcgcagttcatggatgacaatcccgcacagtttcccaagccggactcgggctcctgggtgaacgaactcaaggaagacaacttccttgatggcgacgaaacggctgcgttccgttcggcactcgacatcattggccagcaactgggtaatcagcagagtgacgctggcagtctggcagggacgggtggaggtctgggcactccgagcagtttttccaacaactcgtccgtgatgggtgatccgctgatcgacgccaataccggtcccggtgacagcggcaatacccgtggtgaagcggggcaactgatcggcgagcttatcgaccgtggcctgcaatcggtattggccggtggtggactgggcacacccgtaaacaccccgcagaccggtacgtcggcgaatggcggacagtccgctcaggatcttgatcagttgctgggcggcttgctgctcaagggcctggaggcaacgctcaaggatgccgggcaaacaggcaccgacgtgcagtcgagcgctgcgcaaatcgccaccttgctggtcagtacgctgctgcaaggcacccgcaatcaggctgcagcctga

SEQ ID NO: 9 - protein, 424 aa, Pseudomonas syringae
MSIGITPRPQQTTTPLDFSALSGKSPQPNTFGEQNTQQAIDPSALLFGSDTQKDVNFGTPDSTVQNPQDASKPNDSQSNIAKLISALIMSLLQMLTNSNKKQDTNQEQPDSQAPFQNNGGLGTPSADSGGGGTPDATGGGGGDTPSATGGGGGDTPTATGGGGSGGGGTPTATGGGSGGTPTATGGGEGGVTPQITPQLANPNRTSGTGSVSDTAGSTEQAGKINVVKDTIKVGAGEVFDGHGATFTADKSMGNGDQGENQKPMFELAEGATLKNVNLGENEVDGIHVKAKNAQEVTIDNVHAQNVGEDLITVKGEGGAAVTNLNIKNSSAKGADDKVVQLNANTHLKIDNFKADDFGTMVRTNGGKQFDDMSIELNGIEANHGKFALVKSDSDDLKLATGNIAMTDVKHAYDKTQASTQHTEL

SEQ ID NO: 10 - DNA, 1729 bp, Pseudomonas syringae
tccacttcgctgattttgaaattggcagattcatagaaacgttcaggtgtggaaatcaggctgagtgcgcagatttcgttgataagggtgtggtactggtcattgttggtcatttcaaggcctctgagtgcggtgcggagcaataccagtcttcctgctggcgtgtgcacactgagtcgcaggcataggcatttcagttccttgcgttggttgggcatataaaaaaaggaacttttaaaaacagtgcaatgagatgccggcaaaacgggaaccggtcgctgcgctttgccactcacttcgagcaagctcaaccccaaacatccacatccctatcgaacggacagcgatacggccacttgctctggtaaaccctggagctggcgtcggtccaattgcccacttagcgaggtaacgcagcatgagcatcggcatcacaccccggccgcaacagaccaccacgccactcgatttttcggcgctaagcggcaagagtcctcaaccaaacacgttcggcgagcagaacactcagcaagcgatcgacccgagtgcactgttgttcggcagcgacacacagaaagacgtcaacttcggcacgcccgacagcaccgtccagaatccgcaggacgccagcaagcccaacgacagccagtccaacatcgctaaattgatcagtgcattgatcatgtcgttgctgcagatgctcaccaactccaataaaaagcaggacaccaatcaggaacagcctgatagccaggctcctttccagaacaacggcgggctcggtacaccgtcggccgatagcgggggcggcggtacaccggatgcgacaggtggcggcggcggtgatacgccaagcgcaacaggcggtggcggcggtgatactccgaccgcaacaggcggtggcggcagcggtggcggcggcacacccactgcaacaggtggcggcagcggtggcacacccactgcaacaggcggtggcgagggtggcgtaacaccgcaaatcactccgcagttggccaaccctaaccgtacctcaggtactggctcggtgtcggacaccgcaggttctaccgagcaagccggcaagatcaatgtggtgaaagacaccatcaaggtcggcgctggcgaagtctttgacggccacggcgcaaccttcactgccgacaaatctatgggtaacggagaccagggcgaaaatcagaagcccatgttcgagctggctgaaggcgctacgttgaagaatgtgaacctgggtgagaacgaggtcgatggcatccacgtgaaagccaaaaacgctcaggaagtcaccattgacaacgtgcatgcccagaacgtcggtgaagacctgattacggtcaaaggcgagggaggcgcagcggtcactaatctgaacatcaagaacagcagtgccaaaggtgcagacgacaaggttgtccagctcaacgccaacactcacttgaaaatcgacaacttcaaggccgacgatttcggcacgatggttcgcaccaacggtggcaagcagtttgatgacatgagcatcgagctgaacggcatcgaagctaaccacggcaagttcgccctggtgaaaagcgacagtgacgatctgaagctggcaacgggcaacatcgccatgaccgacgtcaaacacgcctacgataaaacccaggcatcgacccaacacaccgagctttgaatccagacaagtagcttgaaaaaagggggtggactc

SEQ ID NO: 11 - protein, 344 aa, Pseudomonas solanacearum
MSVGNIQSPSNLPGLQNLNLNTNTNSQQSGQSVQDLIKQVEKDILNIIAALVQKAAQSAGGNTGNTGNAPAKDGNANAGANDPSKNDPSKSQAPQSANKTGNVDDANNQDPMQALMQLLEDLVKLLKAALHMQQPGGNDKGNGVGGANGAKGAGGQGGLAEALQEIEQILAQLGGGGAGAGGAGGGVGGAGGADGGSGAGGAGGANGADGGNGVNGNQANGPQNAGDVNGANGADDGSEDQGGLTGVLQKLMKILNALVQMMQQGGLGGGNQAQGGSKGAGNASPASGANPGANQPGSADDQSSGQNNLQSQIMDVVKEVVQILQQMLAAQNGGSQQSTSTQPM

SEQ ID NO: 12 - DNA, 1035 bp, Pseudomonas solanacearum
atgtcagtcggaaacatccagagcccgtcgaacctcccgggtctgcagaacctgaacctcaacaccaacaccaacagccagcaatcgggccagtccgtgcaagacctgatcaagcaggtcgagaaggacatcctcaacatcatcgcagccctcgtgcagaaggccgcacagtcggcgggcggcaacaccggtaacaccggcaacgcgccggcgaaggacggcaatgccaacgcgggcgccaacgacccgagcaagaacgacccgagcaagagccaggctccgcagtcggccaacaagaccggcaacgtcgacgacgccaacaaccaggatccgatgcaagcgctgatgcagctgctggaagacctggtgaagctgctgaaggcggccctgcacatgcagcagcccggcggcaatgacaagggcaacggcgtgggcggtgccaacggcgccaagggtgccggcggccagggcggcctggccgaagcgctgcaggagatcgagcagatcctcgcccagctcggcggcggcggtgctggcgccggcggcgcgggtggcggtgtcggcggtgctggtggcgcggatggcggctccggtgcgggtggcgcaggcggtgcgaacggcgccgacggcggcaatggcgtgaacggcaaccaggcgaacggcccgcagaacgcaggcgatgtcaacggtgccaacggcgcggatgacggcagcgaagaccagggcggcctcaccggcgtgctgcaaaagctgatgaagatcctgaacgcgctggtgcagatgatgcagcaaggcggcctcggcggcggcaaccaggcgcagggcggctcgaagggtgccggcaacgcctcgccggcttccggcgcgaacccgggcgcgaaccagcccggttcggcggatgatcaatcgtccggccagaacaatctgcaatcccagatcatggatgtggtgaaggaggtcgtccagatcctgcagcagatgctggcggcgcagaacggcggcagccagcagtccacctcgacgcagccgatgtaa

SEQ ID NO: 13 - protein, 114 aa, Xanthomonas campestris
MDSIGNNFSNIGNLQTMGIGPQQHEDSSQQSPSAGSEQQLDQLLAMFIMMMLQQSQGSDANQECGNEQPQNGQQEGLSPLTQMLMQIVMQLMQNQGGAGMGGGGSVNSSLGGNA

SEQ ID NO: 14 - DNA, 342 bp, Xanthomonas campestris
atggactctatcggaaacaacttttcgaatatcggcaacctgcagacgatgggcatcgggcctcagcaacacgaggactccagccagcagtcgccttcggctggctccgagcagcagctggatcagttgctcgccatgttcatcatgatgatgctgcaacagagccagggcagcgatgcaaatcaggagtgtggcaacgaacaaccgcagaacggtcaacaggaaggcctgagtccgttgacgcagatgctgatgcagatcgtgatgcagctgatgcagaaccagggcggcgccggcatgggcggtggcggttcggtcaacagcagcctgggcggcaacgcc

SEQ ID NO: 15 - DNA, 342 bp, Nicotiana tabacum; identical to SEQ ID NO: 14

SEQ ID NO: 16 - protein, 35 aa, Nicotiana tabacum
MGFFLFSQMPSFFLVSTLLLFLIISHSSHAQNSRG

(19) United States
(12) Patent Application Publication
(10) Pub. No.: US 2002/0059658 A1
(43) Pub. Date: May 16, 2002

(54) Methods of improving the effectiveness of transgenic plants

(76) Inventors: Zhong-Min Wei, Kirkland, WA (US); Jay Ernest DeRocher, Bothell, WA (US)

Correspondence Address: Michael L. Goldman, NIXON PEABODY LLP, Clinton Square, P.O. Box 31051, Rochester, NY, 14603, US

(21) Appl. No.: 09/880,371

(22) Filed: Jun. 13, 2001

Related U.S. Application Data
(60) Provisional application No. 60/211,585, filed on Jun. 15, 2000.

(51) Int. Cl. A01H 5/00; A01N 25/00

(52) U.S. Cl. 800/278; 800/279; 504/116.1

(57) ABSTRACT

The present invention relates to methods of improving the effectiveness of transgenic plants, either by maximizing the benefit of a transgenic trait in transgenic plants or overcoming deleterious effects on growth, stress tolerance, disease resistance, or insect resistance in transgenic plants expressing a transgenic trait. By applying a hypersensitive response elicitor protein or polypeptide to a transgenic plant expressing a transgene which confers a transgenic trait, or by preparing a transgenic plant expressing both a transgene which confers a transgenic trait and a second transgene which confers hypersensitive response elicitor expression, it is possible to realize the maximum benefit of the transgenic trait or overcome deleterious effects on growth, stress tolerance, disease resistance, or insect resistance which result from or accompany expression of the transgene conferring the transgenic trait.

This application claims benefit of U.S. Provisional Patent Application Serial No. 60/211,585, filed on Jun. 15, 2000, which is hereby incorporated by reference in its entirety.

### FIELD OF THE INVENTION

The present invention relates generally to transgenic plants and methods of improving the effectiveness of transgenic plants either by topical application of a hypersensitive response elicitor to the transgenic plant or by incorporating into the transgenic plant a transgene encoding a hypersensitive response elicitor.

### BACKGROUND OF THE INVENTION

Transfer of genes into plants is an approach being used with increasing frequency to provide useful and advantageous characteristics to crop and ornamental plants that would be difficult or impossible by traditional breeding methods. Transgenic traits can provide the capacity to synthesize specific compounds including vaccines, antibodies, pharmaceutical peptides, plastic, or industrial enzymes or provide improved physical characteristics such as modified fruit ripening, altered fiber properties, enhanced nutrient or dietary fiber content, herbicide resistance, floral color, or better flavor. Other introduced traits are intended to overcome or minimize particular agricultural problems, such as environmental stress, or attack by specific pathogens or pests that prevent maximum yields from being obtained. Transgenic traits that have been commercialized to date have had very specific and limited functions. Many other transgenic traits currently being developed for commercialization or being considered for introduction into crops are similarly limited or specific in their function.

Environmental factors are an important constraint on the yields obtained from transgenic as well as non-transgenic crops. Losses in productivity due to disease and damage caused by pathogens and pests can prevent the full benefit of a transgenic trait from being realized. Since many transgenic traits have no effect on disease or pest resistance, transgenic plants are typically just as susceptible to loss and damage as non-transgenic plants. Transgenic traits designed to confer resistance to pests or disease are, in general, limited in scope—i.e., they are effective only against specific pests or diseases. Such transgenic plants are as vulnerable to non-target pests and diseases as non-transgenic plants. Moreover, the process of introducing a transgenic trait can on occasion result in a crop plant becoming more susceptible to a particular disease. This was observed for some varieties of insect resistant transgenic cotton that lost resistance to a particular fungal pathogen.

Genetically determined inherent growth characteristics of any transgenic plant impose an additional limitation on the potential for benefit to be gained. Transgenic traits being developed for commercialization or that have been commercialized to date do not affect plant growth properties, so efficacy of the traits is restricted by an upper limit on growth even under ideal growing conditions. In some cases it has been observed that the introduction of a transgene conferring a value-added trait can actually cause a reduction in yield. Such a reduction in yield is known as a yield penalty. Yield penalties are tolerated when the value-added trait results in a net economic gain; however, reducing or eliminating the yield penalty would be a clear benefit.

A practical constraint on realizing the maximal benefit from transgenic traits is imposed by the length of time required to develop a transgenic crop to the commercial stage. By the time a transgenic line reaches commercialization, the germplasm used as the starting material may be five or more years old and be at a disadvantage in terms of yield or resistance to specific diseases or pests relative to new germplasms developed in the intervening years. Therefore, it would be desirable to provide an approach that would maximize the benefits of a value-added trait, overcome the yield penalty caused by introduction of a value-added trait, and more rapidly develop a transgenic crop or ornamental lines. To achieve these objectives using existing methods or strategies would be excessively time consuming, technically complex, and without any guarantee of success.

A conventional breeding program is one approach that could be chosen to attempt to obtain a genetic background exhibiting enhanced growth and resistance to diseases and pests into which transgenic traits could be introduced. Unfortunately, achieving even marginal improvements in any one of these characteristics by classical breeding has become increasingly difficult and time consuming as the remaining amount of untapped genetic resources available within a given crop species becomes smaller. There is also no guarantee that this approach is feasible since it is unknown whether achieving useful improvements in all these characteristics simultaneously is possible by conventional breeding.

An alternate approach, at least in principle, would be to introduce into plants, in addition to a gene conferring a desired value-added trait, an array of genes each with a specific resistance or growth enhancement trait to provide an umbrella of resistance and yield improvement effects. A large number of genes have been identified that encode proteins with potential to provide resistance to specific types or classes of pathogens if expressed in transgenic plants. In principle, assembling multiple resistance genes in a transgenic plant could confer resistance to a broad range of pathogens. Such resistance genes, however, would not alter the inherent growth characteristics of the plants. Candidate genes that would serve to enhance overall growth and yield in concert with resistance genes are not obvious. Successfully producing transgenic crops that express arrays of transgenes would be technically complex and require even longer development times than are already needed for generating transgenic plants with a single transgene. Introduction of arrays of transgenes into the same crop plant is an approach yet to be proven in practice.

The use of chemical supplements, including fertilizers and pesticides, to enhance realization of value-added traits is also undesirable due to direct and lingering environmental impact which the chemical supplements can have on water supplies and other organisms in the food chain.

The present invention is directed to overcoming these and other deficiencies in the art.

### SUMMARY OF THE INVENTION

One method of the present invention is carried out by providing a plant or plant seed including a transgene conferring a transgenic trait to the plant or a plant grown from the plant seed, and applying to the plant or plant seed a hypersensitive response elicitor protein or polypeptide. According to one embodiment, the applying of the hypersensitive response elicitor is carried out under conditions effective to impart enhanced growth, stress tolerance, disease resistance, or insect resistance to the plant or the plant grown from the plant seed, thereby maximizing the benefit of the transgenic trait to the plant or the plant grown from the plant seed. According to another embodiment, the transgenic trait is associated with a deleterious effect on growth, stress tolerance, disease resistance, or insect resistance in the transgenic plant and the applying of the hypersensitive response elicitor is carried out under conditions effective to impart enhanced growth, stress tolerance, disease resistance, or insect resistance to the plant or the plant grown from the plant seed, thereby overcoming the deleterious effect.

Another method of the present invention is carried out by providing a plant cell, transforming the plant cell with (i) a first DNA molecule encoding a transcript or a protein or polypeptide which confers a trait to a plant grown from the transformed plant cell and (ii) a second DNA molecule encoding a hypersensitive response elicitor protein or polypeptide which is different than the protein or polypeptide encoded by the first DNA molecule, the transforming being carried out under conditions effective to produce a transformed plant cell, and then regenerating a transgenic plant from the transformed plant cell. According to one embodiment, transforming with the second DNA molecule imparts enhanced growth, stress tolerance, disease resistance, or insect resistance to the plant, thereby maximizing benefit to the plant of the trait conferred by transforming with the first DNA molecule. According to another embodiment, transforming with the first DNA molecule is accompanied by a deleterious effect on growth, stress tolerance, disease resistance, or insect resistance and transforming with the second DNA molecule overcomes the deleterious effect.

Another aspect of the present invention relates to a transgenic plant including a first DNA molecule encoding a transcript or a protein or polypeptide that confers a trait and a second DNA molecule encoding a hypersensitive response elicitor protein or polypeptide different than the protein or polypeptide encoded by the first DNA molecule. Also disclosed is a transgenic plant seed obtained from the transgenic plant of the present invention.

A further aspect of the present invention relates to a system for use in transforming plants with multiple DNA molecules. The system includes a first DNA construct including a first DNA molecule which confers a trait to a host plant and a second DNA construct including a second DNA molecule encoding a hypersensitive response elicitor protein or polypeptide. Also disclosed is an expression system including first and second vectors into which are inserted, respectively, the first and second DNA constructs.

A related aspect of the present invention concerns a DNA construct including a first DNA molecule which confers a trait to a host plant and a second DNA molecule encoding a hypersensitive response elicitor protein or polypeptide. Also disclosed is an expression system including a vector into which is inserted a DNA construct which includes the first and second DNA molecules.

Yet another aspect of the present invention relates to a transgenic host cell including a first DNA molecule encoding a transcript or a protein or polypeptide that confers a trait to a host plant and a second DNA molecule encoding a hypersensitive response elicitor protein or polypeptide which is different than the protein or polypeptide encoded by the first DNA molecule.

The hypersensitive response elicitor, when expressed in or topically applied to transgenic plants, confers a trait of enhanced growth, stress tolerance, broad insect resistance, and broad disease resistance (see WO 96/39802; WO 98/24297; WO 98/32844; and WO 98/37752, which are hereby incorporated by reference in their entirety). By either (i) simultaneously introducing a value-added trait and a trait for hypersensitive response elicitor expression into a plant line or (ii) topically applying a hypersensitive response elicitor to a transgenic plant line expressing a value-added trait, it is possible to obtain a transgenic plant line from which the maximal benefit of the value-added trait can be realized. For example, value-added traits which offer strong but limited benefits (e.g., resistance to a particular pathogen) can be fully realized either by transforming the plants with a transgene or DNA molecule encoding a hypersensitive response elicitor or applying the hypersensitive response elicitor to the plants, both of which will further enhance the same trait by imparting broad growth enhancement, stress tolerance, disease resistance, and/or insect resistance. Similarly, value-added traits which result in a concomitant yield penalty can be fully realized either by transforming the plants with a transgene or DNA molecule encoding a hypersensitive response elicitor or applying the hypersensitive response elicitor to the plants, both of which will overcome the yield penalty by imparting broad growth enhancement, stress tolerance, disease resistance, and/or insect resistance. When expression is utilized rather than topical application, a transgenic germplasm that expresses a hypersensitive response elicitor (i.e., already has enhanced disease resistance and yield properties beyond what is available from conventional hybrid lines) can be transformed with a transgene conferring a specific value-added trait. The same can be said for subsequent introduction of a transgene coding for hypersensitive response elicitor expression into a transgenic germplasm that already expresses a specific value-added trait. Any of these approaches will likely minimize or eliminate any disadvantages relative to conventional hybrids. Thus, the present invention provides an efficient and simple approach which allows for maximal realization of value-added traits and avoids the short-comings and uncertainties of conventional breeding programs.

### DETAILED DESCRIPTION OF THE INVENTION

One aspect of the present invention is a method carried out by providing a plant or plant seed including a transgene conferring a transgenic trait to the plant or a plant grown from the plant seed, and then applying to the plant or plant seed a hypersensitive response elicitor protein or polypeptide. By applying the hypersensitive response elicitor to the plant or plant seed, as discussed infra, enhanced growth, stress tolerance, disease resistance, or insect resistance can be imparted to transgenic plants.

According to one embodiment, the applying of the hypersensitive response elicitor is carried out under conditions effective to impart enhanced growth, stress tolerance, disease resistance, or insect resistance to the plant or the plant grown from the plant seed, thereby maximizing the benefit of the transgenic trait to the plant or the plant grown from the plant seed. For example, when the particular value-added trait relates to specific but limited growth enhancement, stress tolerance, disease resistance, or insect resistance of a transgenic plant, this embodiment relates to providing broad growth enhancement, stress tolerance, disease resistance, or insect resistance that complements the specific but limited value-added trait.

According to another embodiment, the transgenic trait is associated with a deleterious effect on growth, stress tolerance, disease resistance, or insect resistance in the transgenic plant and the applying of the hypersensitive response elicitor is carried out under conditions effective to impart enhanced growth, stress tolerance, disease resistance, or insect resistance to the plant or the plant grown from the plant seed, thereby overcoming the deleterious effect. Thus, this aspect of the present invention is directed to overcoming a yield penalty resulting from a value-added trait.

According to this aspect of the present invention, the effectiveness of a transgenic plant is improved (i.e., maximum benefit is realized or the yield penalty is overcome) following application of a hypersensitive response elicitor protein or polypeptide to either a transgenic plant or a transgenic plant seed from which a plant is grown. The hypersensitive response elicitor protein or polypeptide can be any hypersensitive response elicitor derived from bacterial or fungal sources, although bacterial sources are preferred.

Exemplary hypersensitive response elicitor proteins and polypeptides from bacterial sources include, without limitation, the hypersensitive response elicitors from Erwinia species (e.g., Erwinia amylovora, Erwinia chrysanthemi, Erwinia stewartii, Erwinia carotovora, etc.), Pseudomonas species (e.g., Pseudomonas syringae, Pseudomonas solanacearum, etc.), and Xanthomonas species (e.g., Xanthomonas campestris). In addition to hypersensitive response elicitors from these Gram-negative bacteria, it is possible to use elicitors from Gram-positive bacteria. One example is the hypersensitive response elicitor from Clavibacter michiganensis subsp. sepedonicus.

Exemplary hypersensitive response elicitor proteins or polypeptides from fungal sources include, without limitation, the hypersensitive response elicitors (i.e., elicitins) from various Phytophthora species (e.g., Phytophthora parasitica, Phytophthora cryptogea, Phytophthora cinnamomi, Phytophthora capsici, Phytophthora megasperma, Phytophthora citrophthora, etc.).

The hypersensitive response elicitor protein or polypeptide is derived, preferably, from Erwinia chrysanthemi, Erwinia amylovora, Pseudomonas syringae, or Pseudomonas solanacearum.

A hypersensitive response elicitor protein or polypeptide from Erwinia chrysanthemi has an amino acid sequence corresponding to SEQ. ID. No. 1 as follows:

```
Met Gln Ile Thr Ile Lys Ala His Ile Gly Gly Asp Leu Gly Val Ser
1 5 10 15
Gly Leu Gly Ala Gln Gly Leu Lys Gly Leu Asn Ser Ala Ala Ser Ser
                        20 25 30
Leu Gly Ser Ser Val Asp Lys Leu Ser Ser Thr Ile Asp Lys Leu Thr
                35 40 45
Ser Ala Leu Thr Ser Met Met Phe Gly Gly Ala Leu Ala Gln Gly Leu
        50 55 60
Gly Ala Ser Ser Lys Gly Leu Gly Met Ser Asn Gln Leu Gly Gln Ser
65 70 75 80
Phe Gly Asn Gly Ala Gln Gly Ala Ser Asn Leu Leu Ser Val Pro Lys
                                85 90 95
Ser Gly Gly Asp Ala Leu Ser Lys Met Phe Asp Lys Ala Leu Asp Asp
                        100 105 110
Leu Leu Gly His Asp Thr Val Thr Lys Leu Thr Asn Gln Ser Asn Gln
                115 120 125
Leu Ala Asn Ser Met Leu Asn Ala Ser Gln Met Thr Gln Gly Asn Met
        130 135 140
Asn Ala Phe Gly Ser Gly Val Asn Asn Ala Leu Ser Ser Ile Leu Gly
145 150 155 160
Asn Gly Leu Gly Gln Ser Met Ser Gly Phe Ser Gln Pro Ser Leu Gly
                                165 170 175
Ala Gly Gly Leu Gln Gly Leu Ser Gly Ala Gly Ala Phe Asn Gln Leu
                        180 185 190
Gly Asn Ala Ile Gly Met Gly Val Gly Gln Asn Ala Ala Leu Ser Ala
                195 200 205
Leu Ser Asn Val Ser Thr His Val Asp Gly Asn Asn Arg His Phe Val
        210 215 220
Asp Lys Glu Asp Arg Gly Met Ala Lys Glu Ile Gly Gln Phe Met Asp
225 230 235 240
Gln Tyr Pro Glu Ile Phe Gly Lys Pro Glu Tyr Gln Lys Asp Gly Trp
                                245 250 255
Ser Ser Pro Lys Thr Asp Asp Lys Ser Trp Ala Lys Ala Leu Ser Lys
                        260 265 270
Pro Asp Asp Asp Gly Met Thr Gly Ala Ser Met Asp Lys Phe Arg Gln
                275 280 285
Ala Met Gly Met Ile Lys Ser Ala Val Ala Gly Asp Thr Gly Asn Thr
        290 295 300
Asn Leu Asn Leu Arg Gly Ala Gly Gly Ala Ser Leu Gly Ile Asp Ala
305 310 315 320
Ala Val Val Gly Asp Lys Ile Ala Asn Met Ser Leu Gly Lys Leu Ala
                                325 330 335
Asn Ala
```

This hypersensitive response elicitor protein or polypeptide has a molecular weight of 34 kDa, is heat stable, has a glycine content of greater than 16%, and contains substantially no cysteine. This Erwinia chrysanthemi hypersensitive response elicitor protein or polypeptide is encoded by a DNA molecule having a nucleotide sequence corresponding to SEQ. ID. No. 2 as follows:

| | |
|---|---|
| cgattttacc cgggtgaacg tgctatgacc gacagcatca cggtattcga caccgttacg | 60 |
| gcgtttatgg ccgcgatgaa ccggcatcag gcggcgcgct ggtcgccgca atccggcgtc | 120 |
| gatctggtat ttcagtttgg ggacaccggg cgtgaactca tgatgcagat tcagccgggg | 180 |
| cagcaatatc ccggcatgtt gcgcacgctg ctcgctcgtc gttatcagca ggcggcagag | 240 |
| tgcgatggct gccatctgtg cctgaacggc agcgatgtat tgatcctctg gtggccgctg | 300 |
| ccgtcggatc ccggcagtta tccgcaggtg atcgaacgtt tgtttgaact ggcgggaatg | 360 |
| acgttgccgt cgctatccat agcaccgacg gcgcgtccgc agacagggaa cggacgcgcc | 420 |
| cgatcattaa gataaaggcg gcttttttta ttgcaaaacg gtaacggtga ggaaccgttt | 480 |
| caccgtcggc gtcactcagt aacaagtatc catcatgatg cctacatcgg gatcggcgtg | 540 |
| ggcatccgtt gcagatactt ttgcgaacac ctgacatgaa tgaggaaacg aaattatgca | 600 |
| aattacgatc aaagcgcaca tcggcggtga tttgggcgtc tccggtctgg ggctgggtgc | 660 |
| tcagggactg aaaggactga attccgcggc ttcatcgctg ggttccagcg tggataaact | 720 |
| gagcagcacc atcgataagt tgacctccgc gctgacttcg atgatgtttg gcggcgcgct | 780 |
| ggcgcagggg ctgggcgcca gctcgaaggg gctggggatg agcaatcaac tgggccagtc | 840 |
| tttcggcaat ggcgcgcagg gtgcgagcaa cctgctatcc gtaccgaaat ccggcggcga | 900 |
| tgcgttgtca aaaatgtttg ataaagcgct ggacgatctg ctgggtcatg acaccgtgac | 960 |
| caagctgact aaccagagca accaactggc taattcaatg ctgaacgcca gccagatgac | 1020 |
| ccagggtaat atgaatgcgt tcggcagcgg tgtgaacaac gcactgtcgt ccattctcgg | 1080 |
| caacggtctc ggccagtcga tgagtggctt ctctcagcct tctctggggg caggcggctt | 1140 |
| gcagggcctg agcggcgcgg gtgcattcaa ccagttgggt aatgccatcg gcatgggcgt | 1200 |
| ggggcagaat gctgcgctga gtgcgttgag taacgtcagc acccacgtag acggtaacaa | 1260 |
| ccgccacttt gtagataaag aagatcgcgg catggcgaaa gagatcggcc agtttatgga | 1320 |
| tcagtatccg gaaatattcg gtaaaccgga ataccagaaa gatggctgga gttcgccgaa | 1380 |
| gacggacgac aaatcctggg ctaaagcgct gagtaaaccg gatgatgacg gtatgaccgg | 1440 |
| cgccagcatg gacaaattcc gtcaggcgat gggtatgatc aaaagcgcgg tggcgggtga | 1500 |
| taccggcaat accaacctga acctgcgtgg cgcgggcggt gcatcgctgg gtatcgatgc | 1560 |
| ggctgtcgtc ggcgataaaa tagccaacat gtcgctgggt aagctggcca acgcctgata | 1620 |
| atctgtgctg gcctgataaa gcggaaacga aaaaagagac ggggaagcct gtctcttttc | 1680 |
| ttattatgcg gtttatgcgg ttacctggac cggttaatca tcgtcatcga tctggtacaa | 1740 |
| acgcacattt tcccgttcat tcgcgtcgtt acgcgccaca atcgcgatgg catcttcctc | 1800 |
| gtcgctcaga ttgcgcggct gatggggaac gccgggtgga atatagagaa actcgccggc | 1860 |
| cagatggaga cacgtctgcg ataaatctgt gccgtaacgt gtttctatcc gcccctttag | 1920 |
| cagatagatt gcggtttcgt aatcaacatg gtaatgcggt tccgcctgtg cgccggccgg | 1980 |
| gatcaccaca atattcatag aaagctgtct tgcacctacc gtatcgcggg agataccgac | 2040 |
| aaaatagggc agtttttgcg tggtatccgt ggggtgttcc ggcctgacaa tcttgagttg | 2100 |
| gttcgtcatc atctttctcc atctgggcga cctgatcggt t | 2141 |

The above nucleotide and amino acid sequences are disclosed and further described in U.S. Pat. No. 5,850,015 to Bauer et al. and U.S. Pat. No. 5,776,889 to Wei et al., which are hereby incorporated by reference in their entirety.

A hypersensitive response elicitor protein or polypeptide derived from Erwinia amylovora has an amino acid sequence corresponding to SEQ. ID. No. 3 as follows:

```
Met Ser Leu Asn Thr Ser Gly Leu Gly Ala Ser Thr Met Gln Ile Ser
1 5 10 15
Ile Gly Gly Ala Gly Gly Asn Asn Gly Leu Leu Gly Thr Ser Arg Gln
                        20 25 30
Asn Ala Gly Leu Gly Gly Asn Ser Ala Leu Gly Leu Gly Gly Gly Asn
                35 40 45
Gln Asn Asp Thr Val Asn Gln Leu Ala Gly Leu Leu Thr Gly Met Met
        50 55 60
Met Met Met Ser Met Met Gly Gly Gly Gly Leu Met Gly Gly Gly Leu
65 70 75 80
Gly Gly Gly Leu Gly Asn Gly Leu Gly Gly Ser Gly Gly Leu Gly Glu
                                85 90 95
Gly Leu Ser Asn Ala Leu Asn Asp Met Leu Gly Gly Ser Leu Asn Thr
                        100 105 110
Leu Gly Ser Lys Gly Gly Asn Asn Thr Thr Ser Thr Thr Asn Ser Pro
                115 120 125
Leu Asp Gln Ala Leu Gly Ile Asn Ser Thr Ser Gln Asn Asp Asp Ser
        130 135 140
Thr Ser Gly Thr Asp Ser Thr Ser Asp Ser Ser Asp Pro Met Gln Gln
145 150 155 160
Leu Leu Lys Met Phe Ser Glu Ile Met Gln Ser Leu Phe Gly Asp Gly
                                165 170 175
Gln Asp Gly Thr Gln Gly Ser Ser Ser Gly Gly Lys Gln Pro Thr Glu
                        180 185 190
Gly Glu Gln Asn Ala Tyr Lys Lys Gly Val Thr Asp Ala Leu Ser Gly
                195 200 205
Leu Met Gly Asn Gly Leu Ser Gln Leu Leu Gly Asn Gly Gly Leu Gly
        210 215 220
Gly Gly Gln Gly Gly Asn Ala Gly Thr Gly Leu Asp Gly Ser Ser Leu
225 230 235 240
Gly Gly Lys Gly Leu Gln Asn Leu Ser Gly Pro Val Asp Tyr Gln Gln
                                245 250 255
Leu Gly Asn Ala Val Gly Thr Gly Ile Gly Met Lys Ala Gly Ile Gln
                        260 265 270
Ala Leu Asn Asp Ile Gly Thr His Arg His Ser Ser Thr Arg Ser Phe
                275 280 285
Val Asn Lys Gly Asp Arg Ala Met Ala Lys Glu Ile Gly Gln Phe Met
        290 295 300
Asp Gln Tyr Pro Glu Val Phe Gly Lys Pro Gln Tyr Gln Lys Gly Pro
305 310 315 320
Gly Gln Glu Val Lys Thr Asp Asp Lys Ser Trp Ala Lys Ala Leu Ser
                                325 330 335
Lys Pro Asp Asp Asp Gly Met Thr Pro Ala Ser Met Glu Gln Phe Asn
                        340 345 350
Lys Ala Lys Gly Met Ile Lys Arg Pro Met Ala Gly Asp Thr Gly Asn
                355 360 365
Gly Asn Leu Gln Ala Arg Gly Ala Gly Gly Ser Ser Leu Gly Ile Asp
        370 375 380
Ala Met Met Ala Gly Asp Ala Ile Asn Asn Met Ala Leu Gly Lys Leu
385 390 395 400
Gly Ala Ala
```

This hypersensitive response elicitor protein or polypeptide has a molecular weight of about 39 kDa, has a pI of approximately 4.3, and is heat stable at 100° C. for at least 10 minutes. This hypersensitive response elicitor protein or polypeptide has substantially no cysteine. The hypersensitive response elicitor protein or polypeptide derived from Erwinia amylovora is more fully described in Wei, Z-M., et al., “Harpin, Elicitor of the Hypersensitive Response Produced by the Plant Pathogen Erwinia amylovora,” Science 257:85-88 (1992), which is hereby incorporated by reference in its entirety. The DNA molecule encoding this hypersensitive response elicitor protein or polypeptide has a nucleotide sequence corresponding to SEQ. ID. No. 4 as follows:

| | |
|---|---|
| aagcttcggc atggcacgtt tgaccgttgg gtcggcaggg tacgtttgaa ttattcataa | 60 |
| gaggaatacg ttatgagtct gaatacaagt gggctgggag cgtcaacgat gcaaatttct | 120 |
| atcggcggtg cgggcggaaa taacgggttg ctgggtacca gtcgccagaa tgctgggttg | 180 |
| ggtggcaatt ctgcactggg gctgggcggc ggtaatcaaa atgataccgt caatcagctg | 240 |
| gctggcttac tcaccggcat gatgatgatg atgagcatga tgggcggtgg tgggctgatg | 300 |
| ggcggtggct taggcggtgg cttaggtaat ggcttgggtg gctcaggtgg cctgggcgaa | 360 |
| ggactgtcga acgcgctgaa cgatatgtta ggcggttcgc tgaacacgct gggctcgaaa | 420 |
| ggcggcaaca ataccacttc aacaacaaat tccccgctgg accaggcgct gggtattaac | 480 |
| tcaacgtccc aaaacgacga ttccacctcc ggcacagatt ccacctcaga ctccagcgac | 540 |
| ccgatgcagc agctgctgaa gatgttcagc gagataatgc aaagcctgtt tggtgatggg | 600 |
| caagatggca cccagggcag ttcctctggg ggcaagcagc cgaccgaagg cgagcagaac | 660 |
| gcctataaaa aaggagtcac tgatgcgctg tcgggcctga tgggtaatgg tctgagccag | 720 |
| ctccttggca acgggggact gggaggtggt cagggcggta atgctggcac gggtcttgac | 780 |
| ggttcgtcgc tgggcggcaa agggctgcaa aacctgagcg ggccggtgga ctaccagcag | 840 |
| ttaggtaacg ccgtgggtac cggtatcggt atgaaagcgg gcattcaggc gctgaatgat | 900 |
| atcggtacgc acaggcacag ttcaacccgt tctttcgtca ataaaggcga tcgggcgatg | 960 |
| gcgaaggaaa tcggtcagtt catggaccag tatcctgagg tgtttggcaa gccgcagtac | 1020 |
| cagaaaggcc cgggtcagga ggtgaaaacc gatgacaaat catgggcaaa agcactgagc | 1080 |
| aagccagatg acgacggaat gacaccagcc agtatggagc agttcaacaa agccaagggc | 1140 |
| atgatcaaaa ggcccatggc gggtgatacc ggcaacggca acctgcaggc acgcggtgcc | 1200 |
| ggtggttctt cgctgggtat tgatgccatg atggccggtg atgccattaa caatatggca | 1260 |
| cttggcaagc tgggcgcggc ttaagctt | 1288 |

The above nucleotide and amino acid sequences are disclosed and further described in U.S. Pat. No. 5,849,868 to Beer et al. and U.S. Pat. No. 5,776,889 to Wei et al., which are hereby incorporated by reference in their entirety.

Another hypersensitive response elicitor protein or polypeptide derived from Erwinia amylovora has an amino acid sequence corresponding to SEQ. ID. No. 5 as follows:

```
Met Ser Ile Leu Thr Leu Asn Asn Asn Thr Ser Ser Ser Pro Gly Leu
1 5 10 15
Phe Gln Ser Gly Gly Asp Asn Gly Leu Gly Gly His Asn Ala Asn Ser
                        20 25 30
Ala Leu Gly Gln Gln Pro Ile Asp Arg Gln Thr Ile Glu Gln Met Ala
                35 40 45
Gln Leu Leu Ala Glu Leu Leu Lys Ser Leu Leu Ser Pro Gln Ser Gly
        50 55 60
Asn Ala Ala Thr Gly Ala Gly Gly Asn Asp Gln Thr Thr Gly Val Gly
65 70 75 80
Asn Ala Gly Gly Leu Asn Gly Arg Lys Gly Thr Ala Gly Thr Thr Pro
                                85 90 95
Gln Ser Asp Ser Gln Asn Met Leu Ser Glu Met Gly Asn Asn Gly Leu
                        100 105 110
Asp Gln Ala Ile Thr Pro Asp Gly Gln Gly Gly Gly Gln Ile Gly Asp
                115 120 125
Asn Pro Leu Leu Lys Ala Met Leu Lys Leu Ile Ala Arg Met Met Asp
        130 135 140
Gly Gln Ser Asp Gln Phe Gly Gln Pro Gly Thr Gly Asn Asn Ser Ala
145 150 155 160
Ser Ser Gly Thr Ser Ser Ser Gly Gly Ser Pro Phe Asn Asp Leu Ser
                                165 170 175
Gly Gly Lys Ala Pro Ser Gly Asn Ser Pro Ser Gly Asn Tyr Ser Pro
                        180 185 190
Val Ser Thr Phe Ser Pro Pro Ser Thr Pro Thr Ser Pro Thr Ser Pro
                195 200 205
Leu Asp Phe Pro Ser Ser Pro Thr Lys Ala Ala Gly Gly Ser Thr Pro
        210 215 220
Val Thr Asp His Pro Asp Pro Val Gly Ser Ala Gly Ile Gly Ala Gly
225 230 235 240
Asn Ser Val Ala Phe Thr Ser Ala Gly Ala Asn Gln Thr Val Leu His
                                245 250 255
Asp Thr Ile Thr Val Lys Ala Gly Gln Val Phe Asp Gly Lys Gly Gln
                        260 265 270
Thr Phe Thr Ala Gly Ser Glu Leu Gly Asp Gly Gly Gln Ser Glu Asn
                275 280 285
Gln Lys Pro Leu Phe Ile Leu Glu Asp Gly Ala Ser Leu Lys Asn Val
        290 295 300
Thr Met Gly Asp Asp Gly Ala Asp Gly Ile His Leu Tyr Gly Asp Ala
305 310 315 320
Lys Ile Asp Asn Leu His Val Thr Asn Val Gly Glu Asp Ala Ile Thr
                                325 330 335
Val Lys Pro Asn Ser Ala Gly Lys Lys Ser His Val Glu Ile Thr Asn
                        340 345 350
Ser Ser Phe Glu His Ala Ser Asp Lys Ile Leu Gln Leu Asn Ala Asp
                355 360 365
Thr Asn Leu Ser Val Asp Asn Val Lys Ala Lys Asp Phe Gly Thr Phe
        370 375 380
Val Arg Thr Asn Gly Gly Gln Gln Gly Asn Trp Asp Leu Asn Leu Ser
385 390 395 400
His Ile Ser Ala Glu Asp Gly Lys Phe Ser Phe Val Lys Ser Asp Ser
                                405 410 415
Glu Gly Leu Asn Val Asn Thr Ser Asp Ile Ser Leu Gly Asp Val Glu
                        420 425 430
Asn His Tyr Lys Val Pro Met Ser Ala Asn Leu Lys Val Ala Glu
                435 440 445
```

This protein or polypeptide is acidic, rich in glycine and serine, and lacks cysteine. It is also heat stable, protease sensitive, and suppressed by inhibitors of plant metabolism. The protein or polypeptide of the present invention has a predicted molecular size of ca. 4.5 kDa. The DNA molecule encoding this hypersensitive response elicitor protein or polypeptide has a nucleotide sequence corresponding to SEQ. ID. No. 6 as follows:

| | |
|---|---|
| atgtcaattc ttacgcttaa caacaatacc tcgtcctcgc cgggtctgtt ccagtccggg | 60 |
| ggggacaacg ggcttggtgg tcataatgca aattctgcgt tggggcaaca acccatcgat | 120 |
| cggcaaacca ttgagcaaat ggctcaatta ttggcggaac tgttaaagtc actgctatcg | 180 |
| ccacaatcag gtaatgcggc aaccggagcc ggtggcaatg accagactac aggagttggt | 240 |
| aacgctggcg gcctgaacgg acgaaaaggc acagcaggaa ccactccgca gtctgacagt | 300 |
| cagaacatgc tgagtgagat gggcaacaac gggctggatc aggccatcac gcccgatggc | 360 |
| cagggcggcg ggcagatcgg cgataatcct ttactgaaag ccatgctgaa gcttattgca | 420 |
| cgcatgatgg acggccaaag cgatcagttt ggccaacctg gtacgggcaa caacagtgcc | 480 |
| tcttccggta cttcttcatc tggcggttcc ccttttaacg atctatcagg ggggaaggcc | 540 |
| ccttccggca actccccttc cggcaactac tctcccgtca gtaccttctc acccccatcc | 600 |
| acgccaacgt cccctacctc accgcttgat ttcccttctt ctcccaccaa agcagccggg | 660 |
| ggcagcacgc cggtaaccga tcatcctgac cctgttggta gcgcgggcat cggggccgga | 720 |
| aattcggtgg ccttcaccag cgccggcgct aatcagacgg tgctgcatga caccattacc | 780 |
| gtgaaagcgg gtcaggtgtt tgatggcaaa ggacaaacct tcaccgccgg ttcagaatta | 840 |
| ggcgatggcg gccagtctga aaaccagaaa ccgctgttta tactggaaga cggtgccagc | 900 |
| ctgaaaaacg tcaccatggg cgacgacggg gcggatggta ttcatcttta cggtgatgcc | 960 |
| aaaatagaca atctgcacgt caccaacgtg ggtgaggacg cgattaccgt taagccaaac | 1020 |
| agcgcgggca aaaaatccca cgttgaaatc actaacagtt ccttcgagca cgcctctgac | 1080 |
| aagatcctgc agctgaatgc cgatactaac ctgagcgttg acaacgtgaa ggccaaagac | 1140 |
| tttggtactt ttgtacgcac taacggcggt caacagggta actgggatct gaatctgagc | 1200 |
| catatcagcg cagaagacgg taagttctcg ttcgttaaaa gcgatagcga ggggctaaac | 1260 |
| gtcaatacca gtgatatctc actgggtgat gttgaaaacc actacaaagt gccgatgtcc | 1320 |
| gccaacctga aggtggctga atga | 1344 |

The above nucleotide and amino acid sequences are disclosed and further described in PCT Application Publication No. WO 99/07208 to Kim et al., which is hereby incorporated by reference in its entirety.

A hypersensitive response elicitor protein or polypeptide derived from Pseudomonas syringae has an amino acid sequence corresponding to SEQ. ID. No. 7 as follows:

```
Met Gln Ser Leu Ser Leu Asn Ser Ser Ser Leu Gln Thr Pro Ala Met
1 5 10 15
Ala Leu Val Leu Val Arg Pro Glu Ala Glu Thr Thr Gly Ser Thr Ser
                        20 25 30
Ser Lys Ala Leu Gln Glu Val Val Val Lys Leu Ala Glu Glu Leu Met
                35 40 45
Arg Asn Gly Gln Leu Asp Asp Ser Ser Pro Leu Gly Lys Leu Leu Ala
        50 55 60
Lys Ser Met Ala Ala Asp Gly Lys Ala Gly Gly Gly Ile Glu Asp Val
65 70 75 80
Ile Ala Ala Leu Asp Lys Leu Ile His Glu Lys Leu Gly Asp Asn Phe
                                85 90 95
Gly Ala Ser Ala Asp Ser Ala Ser Gly Thr Gly Gln Gln Asp Leu Met
                        100 105 110
Thr Gln Val Leu Asn Gly Leu Ala Lys Ser Met Leu Asp Asp Leu Leu
                115 120 125
Thr Lys Gln Asp Gly Gly Thr Ser Phe Ser Glu Asp Asp Met Pro Met
        130 135 140
Leu Asn Lys Ile Ala Gln Phe Met Asp Asp Asn Pro Ala Gln Phe Pro
145 150 155 160
Lys Pro Asp Ser Gly Ser Trp Val Asn Glu Leu Lys Glu Asp Asn Phe
                                165 170 175
Leu Asp Gly Asp Glu Thr Ala Ala Phe Arg Ser Ala Leu Asp Ile Ile
                        180 185 190
Gly Gln Gln Leu Gly Asn Gln Gln Ser Asp Ala Gly Ser Leu Ala Gly
                195 200 205
Thr Gly Gly Gly Leu Gly Thr Pro Ser Ser Phe Ser Asn Asn Ser Ser
        210 215 220
Val Met Gly Asp Pro Leu Ile Asp Ala Asn Thr Gly Pro Gly Asp Ser
225 230 235 240
Gly Asn Thr Arg Gly Glu Ala Gly Gln Leu Ile Gly Glu Leu Ile Asp
                                245 250 255
Arg Gly Leu Gln Ser Val Leu Ala Gly Gly Gly Leu Gly Thr Pro Val
                        260 265 270
Asn Thr Pro Gln Thr Gly Thr Ser Ala Asn Gly Gly Gln Ser Ala Gln
                275 280 285
Asp Leu Asp Gln Leu Leu Gly Gly Leu Leu Leu Lys Gly Leu Glu Ala
        290 295 300
Thr Leu Lys Asp Ala Gly Gln Thr Gly Thr Asp Val Gln Ser Ser Ala
305 310 315 320
Ala Gln Ile Ala Thr Leu Leu Val Ser Thr Leu Leu Gln Gly Thr Arg
                                325 330 335
Asn Gln Ala Ala Ala
                        340
```

This hypersensitive response elicitor protein or polypeptide has a molecular weight of 34-35 kDa. It is rich in glycine (about 13.5%) and lacks cysteine and tyrosine. Further information about the hypersensitive response elicitor derived from Pseudomonas syringae is found in He, S. Y., et al., “Pseudomonas syringae pv. syringae Harpin_{Pss}: a Protein that is Secreted via the Hrp Pathway and Elicits the Hypersensitive Response in Plants,” Cell 73:1255-1266 (1993), which is hereby incorporated by reference in its entirety. The DNA molecule encoding this hypersensitive response elicitor from Pseudomonas syringae has a nucleotide sequence corresponding to SEQ. ID. No. 8 as follows:

| | |
|---|---|
| atgcagagtc tcagtcttaa cagcagctcg ctgcaaaccc cggcaatggc ccttgtcctg | 60 |
| gtacgtcctg aagccgagac gactggcagt acgtcgagca aggcgcttca ggaagttgtc | 120 |
| gtgaagctgg ccgaggaact gatgcgcaat ggtcaactcg acgacagctc gccattggga | 180 |
| aaactgttgg ccaagtcgat ggccgcagat ggcaaggcgg gcggcggtat tgaggatgtc | 240 |
| atcgctgcgc tggacaagct gatccatgaa aagctcggtg acaacttcgg cgcgtctgcg | 300 |
| gacagcgcct cgggtaccgg acagcaggac ctgatgactc aggtgctcaa tggcctggcc | 360 |
| aagtcgatgc tcgatgatct tctgaccaag caggatggcg ggacaagctt ctccgaagac | 420 |
| gatatgccga tgctgaacaa gatcgcgcag ttcatggatg acaatcccgc acagtttccc | 480 |
| aagccggact cgggctcctg ggtgaacgaa ctcaaggaag acaacttcct tgatggcgac | 540 |
| gaaacggctg cgttccgttc ggcactcgac atcattggcc agcaactggg taatcagcag | 600 |
| agtgacgctg gcagtctggc agggacgggt ggaggtctgg gcactccgag cagtttttcc | 660 |
| aacaactcgt ccgtgatggg tgatccgctg atcgacgcca ataccggtcc cggtgacagc | 720 |
| ggcaataccc gtggtgaagc ggggcaactg atcggcgagc ttatcgaccg tggcctgcaa | 780 |
| tcggtattgg ccggtggtgg actgggcaca cccgtaaaca ccccgcagac cggtacgtcg | 840 |
| gcgaatggcg gacagtccgc tcaggatctt gatcagttgc tgggcggctt gctgctcaag | 900 |
| ggcctggagg caacgctcaa ggatgccggg caaacaggca ccgacgtgca gtcgagcgct | 960 |
| gcgcaaatcg ccaccttgct ggtcagtacg ctgctgcaag gcacccgcaa tcaggctgca | 1020 |
| gcctga | 1026 |

The above nucleotide and amino acid sequences are disclosed and further described in U.S. Pat. No. 5,708,139 to Collmer et al. and U.S. Pat. No. 5,776,889 to Wei et al., which are hereby incorporated by reference in their entirety.

Another hypersensitive response elicitor protein or polypeptide derived from Pseudomonas syringae has an amino acid sequence corresponding to SEQ. ID. No. 9 as follows:

```
Met Ser Ile Gly Ile Thr Pro Arg Pro Gln Gln Thr Thr Thr Pro Leu
1 5 10 15
Asp Phe Ser Ala Leu Ser Gly Lys Ser Pro Gln Pro Asn Thr Phe Gly
                        20 25 30
Glu Gln Asn Thr Gln Gln Ala Ile Asp Pro Ser Ala Leu Leu Phe Gly
                35 40 45
Ser Asp Thr Gln Lys Asp Val Asn Phe Gly Thr Pro Asp Ser Thr Val
        50 55 60
Gln Asn Pro Gln Asp Ala Ser Lys Pro Asn Asp Ser Gln Ser Asn Ile
65 70 75 80
Ala Lys Leu Ile Ser Ala Leu Ile Met Ser Leu Leu Gln Met Leu Thr
                                85 90 95
Asn Ser Asn Lys Lys Gln Asp Thr Asn Gln Glu Gln Pro Asp Ser Gln
                        100 105 110
Ala Pro Phe Gln Asn Asn Gly Gly Leu Gly Thr Pro Ser Ala Asp Ser
                115 120 125
Gly Gly Gly Gly Thr Pro Asp Ala Thr Gly Gly Gly Gly Gly Asp Thr
        130 135 140
Pro Ser Ala Thr Gly Gly Gly Gly Gly Asp Thr Pro Thr Ala Thr Gly
145 150 155 160
Gly Gly Gly Ser Gly Gly Gly Gly Thr Pro Thr Ala Thr Gly Gly Gly
                                165 170 175
Ser Gly Gly Thr Pro Thr Ala Thr Gly Gly Gly Glu Gly Gly Val Thr
                        180 185 190
Pro Gln Ile Thr Pro Gln Leu Ala Asn Pro Asn Arg Thr Ser Gly Thr
                195 200 205
Gly Ser Val Ser Asp Thr Ala Gly Ser Thr Glu Gln Ala Gly Lys Ile
        210 215 220
Asn Val Val Lys Asp Thr Ile Lys Val Gly Ala Gly Glu Val Phe Asp
225 230 235 240
Gly His Gly Ala Thr Phe Thr Ala Asp Lys Ser Met Gly Asn Gly Asp
                                245 250 255
Gln Gly Glu Asn Gln Lys Pro Met Phe Glu Leu Ala Glu Gly Ala Thr
                        260 265 270
Leu Lys Asn Val Asn Leu Gly Glu Asn Glu Val Asp Gly Ile His Val
                275 280 285
Lys Ala Lys Asn Ala Gln Glu Val Thr Ile Asp Asn Val His Ala Gln
        290 295 300
Asn Val Gly Glu Asp Leu Ile Thr Val Lys Gly Glu Gly Gly Ala Ala
305 310 315 320
Val Thr Asn Leu Asn Ile Lys Asn Ser Ser Ala Lys Gly Ala Asp Asp
                                325 330 335
Lys Val Val Gln Leu Asn Ala Asn Thr His Leu Lys Ile Asp Asn Phe
                        340 345 350
Lys Ala Asp Asp Phe Gly Thr Met Val Arg Thr Asn Gly Gly Lys Gln
                355 360 365
Phe Asp Asp Met Ser Ile Glu Leu Asn Gly Ile Glu Ala Asn His Gly
        370 375 380
Lys Phe Ala Leu Val Lys Ser Asp Ser Asp Asp Leu Lys Leu Ala Thr
385 390 395 400
Gly Asn Ile Ala Met Thr Asp Val Lys His Ala Tyr Asp Lys Thr Gln
                                405 410 415
Ala Ser Thr Gln His Thr Glu Leu
                        420
```

This protein or polypeptide is acidic, glycine-rich, lacks cysteine, and is deficient in aromatic amino acids. The DNA molecule encoding this hypersensitive response elicitor from Pseudomonas syringae has a nucleotide sequence corresponding to SEQ. ID. No. 10 as follows:

| | |
|---|---|
| tccacttcgc tgattttgaa attggcagat tcatagaaac gttcaggtgt ggaaatcagg | 60 |
| ctgagtgcgc agatttcgtt gataagggtg tggtactggt cattgttggt catttcaagg | 120 |
| cctctgagtg cggtgcggag caataccagt cttcctgctg gcgtgtgcac actgagtcgc | 180 |
| aggcataggc atttcagttc cttgcgttgg ttgggcatat aaaaaaagga acttttaaaa | 240 |
| acagtgcaat gagatgccgg caaaacggga accggtcgct gcgctttgcc actcacttcg | 300 |
| agcaagctca accccaaaca tccacatccc tatcgaacgg acagcgatac ggccacttgc | 360 |
| tctggtaaac cctggagctg gcgtcggtcc aattgcccac ttagcgaggt aacgcagcat | 420 |
| gagcatcggc atcacacccc ggccgcaaca gaccaccacg ccactcgatt tttcggcgct | 480 |
| aagcggcaag agtcctcaac caaacacgtt cggcgagcag aacactcagc aagcgatcga | 540 |
| cccgagtgca ctgttgttcg gcagcgacac acagaaagac gtcaacttcg gcacgcccga | 600 |
| cagcaccgtc cagaatccgc aggacgccag caagcccaac gacagccagt ccaacatcgc | 660 |
| taaattgatc agtgcattga tcatgtcgtt gctgcagatg ctcaccaact ccaataaaaa | 720 |
| gcaggacacc aatcaggaac agcctgatag ccaggctcct ttccagaaca acggcgggct | 780 |
| cggtacaccg tcggccgata gcgggggcgg cggtacaccg gatgcgacag gtggcggcgg | 840 |
| cggtgatacg ccaagcgcaa caggcggtgg cggcggtgat actccgaccg caacaggcgg | 900 |
| tggcggcagc ggtggcggcg gcacacccac tgcaacaggt ggcggcagcg gtggcacacc | 960 |
| cactgcaaca ggcggtggcg agggtggcgt aacaccgcaa atcactccgc agttggccaa | 1020 |
| ccctaaccgt acctcaggta ctggctcggt gtcggacacc gcaggttcta ccgagcaagc | 1080 |
| cggcaagatc aatgtggtga aagacaccat caaggtcggc gctggcgaag tctttgacgg | 1140 |
| ccacggcgca accttcactg ccgacaaatc tatgggtaac ggagaccagg gcgaaaatca | 1200 |
| gaagcccatg ttcgagctgg ctgaaggcgc tacgttgaag aatgtgaacc tgggtgagaa | 1260 |
| cgaggtcgat ggcatccacg tgaaagccaa aaacgctcag gaagtcacca ttgacaacgt | 1320 |
| gcatgcccag aacgtcggtg aagacctgat tacggtcaaa ggcgagggag gcgcagcggt | 1380 |
| cactaatctg aacatcaaga acagcagtgc caaaggtgca gacgacaagg ttgtccagct | 1440 |
| caacgccaac actcacttga aaatcgacaa cttcaaggcc gacgatttcg gcacgatggt | 1500 |
| tcgcaccaac ggtggcaagc agtttgatga catgagcatc gagctgaacg gcatcgaagc | 1560 |
| taaccacggc aagttcgccc tggtgaaaag cgacagtgac gatctgaagc tggcaacggg | 1620 |
| caacatcgcc atgaccgacg tcaaacacgc ctacgataaa acccaggcat cgacccaaca | 1680 |
| caccgagctt tgaatccaga caagtagctt gaaaaaaggg ggtggactc | 1729 |

The above nucleotide and amino acid sequences are disclosed and further described in U.S. Pat. No. 6,172,184 to Collmer et al., which is hereby incorporated by reference in its entirety.

A hypersensitive response elicitor protein or polypeptide derived from Pseudomonas solanacearum has an amino acid sequence corresponding to SEQ. ID. No. 11 as follows:

```
Met Ser Val Gly Asn Ile Gln Ser Pro Ser Asn Leu Pro Gly Leu Gln
1 5 10 15
Asn Leu Asn Leu Asn Thr Asn Thr Asn Ser Gln Gln Ser Gly Gln Ser
                        20 25 30
Val Gln Asp Leu Ile Lys Gln Val Glu Lys Asp Ile Leu Asn Ile Ile
                35 40 45
Ala Ala Leu Val Gln Lys Ala Ala Gln Ser Ala Gly Gly Asn Thr Gly
        50 55 60
Asn Thr Gly Asn Ala Pro Ala Lys Asp Gly Asn Ala Asn Ala Gly Ala
65 70 75 80
Asn Asp Pro Ser Lys Asn Asp Pro Ser Lys Ser Gln Ala Pro Gln Ser
                                85 90 95
Ala Asn Lys Thr Gly Asn Val Asp Asp Ala Asn Asn Gln Asp Pro Met
                        100 105 110
Gln Ala Leu Met Gln Leu Leu Glu Asp Leu Val Lys Leu Leu Lys Ala
                115 120 125
Ala Leu His Met Gln Gln Pro Gly Gly Asn Asp Lys Gly Asn Gly Val
        130 135 140
Gly Gly Ala Asn Gly Ala Lys Gly Ala Gly Gly Gln Gly Gly Leu Ala
145 150 155 160
Glu Ala Leu Gln Glu Ile Glu Gln Ile Leu Ala Gln Leu Gly Gly Gly
                                165 170 175
Gly Ala Gly Ala Gly Gly Ala Gly Gly Gly Val Gly Gly Ala Gly Gly
                        180 185 190
Ala Asp Gly Gly Ser Gly Ala Gly Gly Ala Gly Gly Ala Asn Gly Ala
                195 200 205
Asp Gly Gly Asn Gly Val Asn Gly Asn Gln Ala Asn Gly Pro Gln Asn
        210 215 220
Ala Gly Asp Val Asn Gly Ala Asn Gly Ala Asp Asp Gly Ser Glu Asp
225 230 235 240
Gln Gly Gly Leu Thr Gly Val Leu Gln Lys Leu Met Lys Ile Leu Asn
                                245 250 255
Ala Leu Val Gln Met Met Gln Gln Gly Gly Leu Gly Gly Gly Asn Gln
                        260 265 270
Ala Gln Gly Gly Ser Lys Gly Ala Gly Asn Ala Ser Pro Ala Ser Gly
                275 280 285
Ala Asn Pro Gly Ala Asn Gln Pro Gly Ser Ala Asp Asp Gln Ser Ser
        290 295 300
Gly Gln Asn Asn Leu Gln Ser Gln Ile Met Asp Val Val Lys Glu Val
305 310 315 320
Val Gln Ile Leu Gln Gln Met Leu Ala Ala Gln Asn Gly Gly Ser Gln
                                325 330 335
Gln Ser Thr Ser Thr Gln Pro Met
                        340
```

Further information regarding this hypersensitive response elicitor protein or polypeptide derived from Pseudomonas solanacearum is set forth in Arlat, M., et al., “PopA1, a Protein which Induces a Hypersensitive-like Response in Specific Petunia Genotypes, is Secreted via the Hrp Pathway of Pseudomonas solanacearum,” EMBO J. 13:543-533 (1994), which is hereby incorporated by reference in its entirety. It is encoded by a DNA molecule from Pseudomonas solanacearum having a nucleotide sequence corresponding SEQ. ID. No. 12 as follows:

| | |
|---|---|
| atgtcagtcg gaaacatcca gagcccgtcg aacctcccgg gtctgcagaa cctgaacctc | 60 |
| aacaccaaca ccaacagcca gcaatcgggc cagtccgtgc aagacctgat caagcaggtc | 120 |
| gagaaggaca tcctcaacat catcgcagcc ctcgtgcaga aggccgcaca gtcggcgggc | 180 |
| ggcaacaccg gtaacaccgg caacgcgccg gcgaaggacg gcaatgccaa cgcgggcgcc | 240 |
| aacgacccga gcaagaacga cccgagcaag agccaggctc cgcagtcggc caacaagacc | 300 |
| ggcaacgtcg acgacgccaa caaccaggat ccgatgcaag cgctgatgca gctgctggaa | 360 |
| gacctggtga agctgctgaa ggcggccctg cacatgcagc agcccggcgg caatgacaag | 420 |
| ggcaacggcg tgggcggtgc caacggcgcc aagggtgccg gcggccaggg cggcctggcc | 480 |
| gaagcgctgc aggagatcga gcagatcctc gcccagctcg gcggcggcgg tgctggcgcc | 540 |
| ggcggcgcgg gtggcggtgt cggcggtgct ggtggcgcgg atggcggctc cggtgcgggt | 600 |
| ggcgcaggcg gtgcgaacgg cgccgacggc ggcaatggcg tgaacggcaa ccaggcgaac | 660 |
| ggcccgcaga acgcaggcga tgtcaacggt gccaacggcg cggatgacgg cagcgaagac | 720 |
| cagggcggcc tcaccggcgt gctgcaaaag ctgatgaaga tcctgaacgc gctggtgcag | 780 |
| atgatgcagc aaggcggcct cggcggcggc aaccaggcgc agggcggctc gaagggtgcc | 840 |
| ggcaacgcct cgccggcttc cggcgcgaac ccgggcgcga accagcccgg ttcggcggat | 900 |
| gatcaatcgt ccggccagaa caatctgcaa tcccagatca tggatgtggt gaaggaggtc | 960 |
| gtccagatcc tgcagcagat gctggcggcg cagaacggcg gcagccagca gtccacctcg | 1020 |
| acgcagccga tgtaa | 1035 |

The above nucleotide and amino acid sequences are disclosed and further described in U.S. Pat. No. 5,776,889 to Wei et al., which is hereby incorporated by reference in its entirety.

A hypersensitive response elicitor polypeptide or protein derived from Xanthomonas campestris has an amino acid sequence corresponding to SEQ. ID. No. 13 as follows:

```
Met Asp Ser Ile Gly Asn Asn Phe Ser Asn Ile Gly Asn Leu Gln Thr
    1 5 10 15
Met Gly Ile Gly Pro Gln Gln His Glu Asp Ser Ser Gln Gln Ser Pro
                          20 25 30
Ser Ala Gly Ser Glu Gln Gln Leu Asp Gln Leu Leu Ala Met Phe Ile
                  35 40 45
Met Met Met Leu Gln Gln Ser Gln Gly Ser Asp Ala Asn Gln Glu Cys
          50 55 60
Gly Asn Glu Gln Pro Gln Asn Gly Gln Gln Glu Gly Leu Ser Pro Leu
  65 70 75 80
Thr Gln Met Leu Met Gln Ile Val Met Gln Leu Met Gln Asn Gln Gly
                                  85 90 95
Gly Ala Gly Met Gly Gly Gly Gly Ser Val Asn Ser Ser Leu Gly Gly
                        100 105 110
Asn Ala
```

This hypersensitive response elicitor polypeptide or protein has an estimated molecular weight of about 12 kDa based on the deduced amino acid sequence, which is consistent with a molecular weight of about 14 kDa as detected by SDS-PAGE. The above protein or polypeptide is encoded by a DNA molecule according to SEQ. ID. No. 14 as follows:

| | |
|---|---|
| atggactcta tcggaaacaa cttttcgaat atcggcaacc tgcagacgat gggcatcggg | 60 |
| cctcagcaac acgaggactc cagccagcag tcgccttcgg ctggctccga gcagcagctg | 120 |
| gatcagttgc tcgccatgtt catcatgatg atgctgcaac agagccaggg cagcgatgca | 180 |
| aatcaggagt gtggcaacga acaaccgcag aacggtcaac aggaaggcct gagtccgttg | 240 |
| acgcagatgc tgatgcagat cgtgatgcag ctgatgcaga accagggcgg cgccggcatg | 300 |
| ggcggtggcg gttcggtcaa cagcagcctg ggcggcaacg cc | 342 |

The above nucleotide and amino acid sequences are disclosed and further described in U.S. patent application Ser. No. 09/829,124, which is hereby incorporated by reference in its entirety.

Other embodiments of the present invention include, but are not limited to, use of a hypersensitive response elicitor protein or polypeptide derived from Erwinia carotovora and Erwinia stewartii. Isolation of Erwinia carotovora hypersensitive response elicitor protein or polypeptide is described in Cui, et al., “The RsmA Mutants of Erwinia carotovora subsp. carotovora Strain Ecc71 Overexpress hrp N_{Ecc} and Elicit a Hypersensitive Reaction-like Response in Tobacco Leaves,” MPMI, 9(7):565-73 (1996), which is hereby incorporated by reference in its entirety. A hypersensitive response elicitor protein or polypeptide of Erwinia stewartii is set forth in Ahmad, et al., “Harpin is Not Necessary for the Pathogenicity of Erwinia stewartii on Maize,” 8th Int'l. Cong. Molec. Plant-Microbe Interact., Jul. 14-19, 1996 and Ahmad, et al., “Harpin is Not Necessary for the Pathogenicity of Erwinia stewartii on Maize,” Ann. Mtg. Am. Phytopath. Soc., Jul. 27-31, 1996, which are hereby incorporated by reference in their entirety.

The hypersensitive response elicitor proteins or polypeptides from various Phytophthora species are described in Kaman, et al., “Extracellular Protein Elicitors from Phytophthora: Most Specificity and Induction of Resistance to Bacterial and Fungal Phytopathogens,” Molec. Plant-Microbe Interact., 6(1):15-25 (1993); Ricci, et al., “Structure and Activity of Proteins from Pathogenic Fungi Phytophthora Eliciting Necrosis and Acquired Resistance in Tobacco,” Eur. J. Biochem., 183:555-63 (1989); Ricci, et al., “Differential Production of Parasiticein, and Elicitor of Necrosis and Resistance in Tobacco, by Isolates of Phytophthora parasitica,” Plant Path. 41:298-307 (1992); Baillreul, et al., “A New Elicitor of the Hypersensitive Response in Tobacco: A Fungal Glycoprotein Elicits Cell Death, Expression of Defense Genes, Production of Salicylic Acid, and Induction of Systemic Acquired Resistance,” Plant J., 8(4):551-60 (1995), and Bonnet, et al., “Acquired Resistance Triggered by Elicitors in Tobacco and Other Plants,” Eur. J. Plant Path., 102:181-92 (1996), which are hereby incorporated by reference in their entirety.

Another hypersensitive response elicitor for use in accordance with the present invention is derived from Clavibacter michiganensis subsp. sepedonicus. The use of this particular hypersensitive response elicitor is described in U.S. patent application Ser. No. 09/136,625, which is hereby incorporated by reference in its entirety.

Other elicitors can be readily identified by isolating putative hypersensitive response elicitors and testing them for elicitor activity as described, for example, in Wei, Z-M., et al., “Harpin, Elicitor of the Hypersensitive Response Produced by the Plant Pathogen Erwinia amylovora,” Science 257:85-88 (1992), which is hereby incorporated by reference in its entirety. Cell-free preparations from culture supernatants can be tested for elicitor activity (i.e., local necrosis) by using them to infiltrate appropriate plant tissues. Once identified, DNA molecules encoding a hypersensitive response elicitor can be isolated using standard techniques known to those skilled in the art.

The hypersensitive response elicitor protein or polypeptide can also be a fragment of the above hypersensitive response elicitor proteins or polypeptides as well as fragments of full length elicitors from other pathogens.

Suitable fragments can be produced by several means. Subclones of the gene encoding a known elicitor protein can be produced using conventional molecular genetic manipulation for subcloning gene fragments, such as described by Sambrook et al., Molecular Cloning: A Laboratory Manual, Cold Springs Laboratory, Cold Springs Harbor, N.Y. (1989), and Ausubel et al. (ed.), Current Protocols in Molecular Biology, John Wiley & Sons (New York, N.Y.) (1999 and preceding editions), which are hereby incorporated by reference in their entirety. The subclones then are expressed in vitro or in vivo in bacterial cells to yield a smaller protein or polypeptide that can be tested for elicitor activity, e.g., using procedures set forth in Wei, Z-M., et al., Science 257: 85-88 (1992), which is hereby incorporated by reference in its entirety.

In another approach, based on knowledge of the primary structure of the protein, fragments of the elicitor protein gene may be synthesized using the PCR technique together with specific sets of primers chosen to represent particular portions of the protein. Erlich, H. A., et al., “Recent Advances in the Polymerase Chain Reaction,” Science 252:1643-51 (1991), which is hereby incorporated by reference in its entirety. These can then be cloned into an appropriate vector for expression of a truncated protein or polypeptide from bacterial cells as described above.

An example of suitable fragments of a hypersensitive response elicitor which elicit a hypersensitive response are fragments of the Erwinia amylovora hypersensitive response elicitor protein or polypeptide of SEQ. ID. No. 3. The fragments can be a C-terminal fragment of the amino acid sequence of SEQ. ID. No. 3, an N-terminal fragment of the amino acid sequence of SEQ. ID. No. 3, or an internal fragment of the amino acid sequence of SEQ. ID. No. 3. The C-terminal fragment of the amino acid sequence of SEQ. ID. No. 3 can span amino acids 105 and 403 of SEQ. ID. No. 3. The N-terminal fragment of the amino acid sequence of SEQ. ID. No. 3 can span the following amino acids of SEQ. ID. No. 3: 1 and 98, 1 and 104, 1 and 122, 1 and 168, 1 and 218, 1 and 266, 1 and 342, 1 and 321, and 1 and 372. The internal fragment of the amino acid sequence of SEQ. ID. No. 3 can span the following amino acids of SEQ. ID. No. 3: 76 and 209, 105 and 209, 99 and 209, 137 and 204, 137 and 200, 109 and 204, 109 and 200, 137 and 180, and 105 and 180. DNA molecules encoding these fragments can also be utilized in the chimeric gene of the present invention.

DNA molecules encoding a hypersensitive response elicitor protein or polypeptide can also include a DNA molecule that hybridizes under stringent conditions to the DNA molecule having nucleotide sequence of SEQ. ID. Nos. 2, 4, 6, 8, 10, 12, or 14. An example of suitable stringency conditions is when hybridization is carried out at a temperature of about 37° C. using a hybridization medium that includes 0.9M sodium citrate (“SSC”) buffer, followed by washing with 0.2× SSC buffer at 37° C. Higher stringency can readily be attained by increasing the temperature for either hybridization or washing conditions or increasing the sodium concentration of the hybridization or wash medium. Nonspecific binding may also be controlled using any one of a number of known techniques such as, for example, blocking the membrane with protein-containing solutions, addition of heterologous RNA, DNA, and SDS to the hybridization buffer, and treatment with RNase. Wash conditions are typically performed at or below stringency. Exemplary high stringency conditions include carrying out hybridization at a temperature of about 42° C. to about 65° C. for up to about 20 hours in a hybridization medium containing 1M NaCl, 50 mM Tris-HCl, pH 7.4, 10 mM EDTA, 0.1% sodium dodecyl sulfate (SDS), 0.2% ficoll, 0.2% polyvinylpyrrolidone, 0.2% bovine serum albumin, and 50 μg/ml E. coli DNA, followed by washing carried out at between about 42° C. to about 65° C. in a 0.2× SSC buffer.

Variants of suitable hypersensitive response elicitor proteins or polypeptides can also be expressed. Variants may be made by, for example, the deletion, addition, or alteration of amino acids that have minimal influence on the properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification, or identification of the polypeptide.

When it is desirable to perform the methods of the present invention with application of the hypersensitive response elicitor protein or polypeptide to a plant seed or a plant, it is preferable, though not necessary, that the hypersensitive response elicitor protein or polypeptide be applied in isolated form or with a carrier as discussed hereinafter.

One particular hypersensitive response elicitor protein, known as harpin_{Ea} is commercially available from Eden Bioscience Corporation (Bothell, Wash.) under the name of Messenger®. Messenger® contains 3% by weight of harpin_{Ea} as the active ingredient and 97% by weight inert ingredients. Harpin_{Ea} is one type of hypersensitive response elicitor protein from Erwinia amylovora, identified herein by SEQ. ID. No. 3.

Alternatively, the hypersensitive response elicitor protein or polypeptide can be recombinantly produced, isolated, and then purified, if necessary. When recombinantly produced, the hypersensitive response elicitor protein or polypeptide is expressed in a recombinant host cell, typically, although not exclusively, a prokaryote.

When a prokaryotic host cell is selected for subsequent transformation, the promoter region used to construct the recombinant DNA molecule (i.e., transgene) should be appropriate for the particular host. The DNA sequences of eukaryotic promoters, as described infra for plants, differ from those of prokaryotic promoters. Eukaryotic promoters and accompanying genetic signals may not be recognized in or may not function in a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology, 68:473 (1979), which is hereby incorporated by reference in its entirety.

Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals. For instance, efficient translation in E. coli requires a Shine-Dalgarno (“SD”) sequence about 7-9 bases 5′ to the initiation codon (“ATG”) to provide a ribosome binding site. Thus, any SD-ATG combination that can be utilized by host cell ribosomes may be employed. Such combinations include, but are not limited to, the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

Once the DNA molecule coding for a hypersensitive response elicitor protein or polypeptide has been ligated to its appropriate regulatory regions using well known molecular cloning techniques, it can then be introduced into a vector or otherwise introduced directly into a host cell (Sambrook et al., Molecular Cloning: A Laboratory Manual, Second Edition, Cold Spring Harbor Press, NY (1989), which is hereby incorporated by reference in its entirety).

The recombinant molecule can be introduced into host cells via transformation, particularly transduction, conjugation, mobilization, or electroporation. Suitable host cells include, but are not limited to, bacteria, virus, yeast, mammalian cells, insect, plant, and the like. Preferably the host cells are either a bacterial cell or a plant cell. The host cells, when grown in an appropriate medium, are capable of expressing the hypersensitive response elicitor protein or polypeptide, which can then be isolated therefrom and, if necessary, purified.

The hypersensitive response elicitor protein or polypeptide of the present invention is preferably produced in purified form (preferably at least about 60%, more preferably 80%, pure) by conventional techniques. Typically, the protein or polypeptide of the present invention is produced but not secreted into the growth medium of recombinant host cells, usually although not exclusively bacterial host cells. Alternatively, the protein or polypeptide of the present invention is secreted into growth medium.

In the case of an unsecreted hypersensitive response elicitor protein or polypeptide, the protein or polypeptide can be isolated from the host cell (e.g., E. coli) carrying a recombinant plasmid by lysing the host cell with sonication, heat, or chemical treatment, after which the homogenate is centrifuged to remove bacterial debris. The supernatant is then subjected to heat treatment and the hypersensitive response elicitor is separated by centrifugation. The supernatant fraction containing the hypersensitive response elicitor protein is subjected to gel filtration in an appropriately sized dextran or polyacrylamide column to separate the proteins. If necessary, the protein fraction may be further purified by ion exchange or HPLC.

Alternatively, it is desirable for recombinant host cells to secrete the hypersensitive response elicitor protein or polypeptide into growth medium, thereby avoiding the need to lyse cells and remove cellular debris. To enable the host cell to secrete the hypersensitive response elicitor, the host cell can also be transformed with a type III secretion system in accordance with Ham et al., “A Cloned Erwinia chrysanthemi Hrp (Type III Protein Secretion) System Functions in Escherichia coli to Deliver Pseudomonas syringae Avr Signals to Plant Cells and Secrete Avr Proteins in Culture,” Microbiol. 95:10206-10211 (1998), which is hereby incorporated by reference in its entirety. After growing recombinant host cells which secrete the hypersensitive response elicitor into growth medium, isolation of the hypersensitive response elicitor protein or polypeptide from growth medium can be carried out substantially as described above.

The methods of the present invention which involve application of the hypersensitive response elicitor polypeptide or protein can be carried out through a variety of procedures when all or part of the plant is treated, including leaves, stems, roots, etc. This may (but need not) involve infiltration of the hypersensitive response elicitor polypeptide or protein into the plant. Suitable application methods include high or low pressure spraying, injection, dusting, and leaf abrasion proximate to when elicitor application takes place. More than one application of the hypersensitive response elicitor protein or polypeptide may be desirable either to realize maximal benefit of the value-added trait or overcome a yield penalty, particularly over the course of a growing season. When treating plant seeds in accordance with the application embodiment of the present invention, the hypersensitive response elicitor protein or polypeptide can be applied by low or high pressure spraying, coating, immersion, dusting, or injection. Other suitable application procedures can be envisioned by those skilled in the art provided they are able to effect contact of the hypersensitive response elicitor polypeptide or protein with cells of the plant or plant seed. Once treated with the hypersensitive response elicitor of the present invention, the seeds can be planted in natural or artificial soil and cultivated using conventional procedures to produce plants. After plants have been propagated from seeds treated in accordance with the present invention, the plants may also be treated with one or more applications of the hypersensitive response elicitor protein or polypeptide. Such propagated plants may, in turn, be useful in producing seeds or propagules (e.g., cuttings) that produce plants capable of insect control.

The hypersensitive response elicitor polypeptide or protein can be applied to plants or plant seeds in accordance with the present invention alone or in a mixture with other materials. Alternatively, the hypersensitive response elicitor polypeptide or protein can be applied separately to plants with other materials being applied at different times.

A composition suitable for treating plants or plant seeds in accordance with the application embodiment of the present invention contains a hypersensitive response elicitor polypeptide or protein in a carrier. Suitable carriers include water, aqueous solutions, slurries, or dry powders. In this embodiment, the composition contains greater than 500 nM hypersensitive response elicitor polypeptide or protein.

Although not required, this composition may contain additional additives including fertilizer, insecticide, fungicide, nematacide, herbicide, and mixtures thereof. Suitable fertilizers include (NH₄)₂NO₃. An example of a suitable insecticide is Malathion. Useful fungicides include Captan.

Other suitable additives include buffering agents, wetting agents, coating agents, and abrading agents. These materials can be used to facilitate the process of the present invention. In addition, the hypersensitive response elicitor polypeptide or protein can be applied to plant seeds with other conventional seed formulation and treatment materials, including clays and polysaccharides.

Although application of the hypersensitive response elicitor protein or polypeptide is preferably carried out in isolated form or with a carrier, the hypersensitive response elicitor protein or polypeptide can also be applied in a non-isolated but non-infectious form. When applied in non-isolated but non-infectious form, the hypersensitive response elicitor is applied indirectly to the plant via application of a bacteria which expresses and then secretes or injects the expressed hypersensitive response elicitor protein or polypeptide into plant cells or tissues. Such application can be carried out by applying the bacteria to all or part of a plant or a plant seed under conditions where the polypeptide or protein contacts all or part of the cells of the plant or plant seed. Alternatively, the hypersensitive response elicitor protein or polypeptide can be applied to plants such that seeds recovered from such plants themselves are able to enhance plant growth, impart stress tolerance in plants, impart disease resistance in plants, and/or to effect insect control.

The embodiment of the present invention where the hypersensitive response elicitor polypeptide or protein is applied to the plant or plant seed in a non-isolated but non-infectious form can be carried out in a number of ways, including: 1) application of bacteria which do not cause disease and are transformed with genes encoding a hypersensitive response elicitor polypeptide or protein, and 2) application of bacteria which cause disease in some plant species (but not in those to which they are applied) and naturally contain a gene encoding the hypersensitive response elicitor polypeptide or protein.

In one embodiment of the bacterial application mode of the present invention, the bacteria do not cause disease and have been transformed (e.g., recombinantly) with genes encoding a hypersensitive response elicitor polypeptide or protein. For example, E. coli, which does not elicit a hypersensitive response in plants, can be transformed with genes encoding a hypersensitive response elicitor polypeptide or protein and then applied to plants. Bacterial species other than E. coli can also be used in this embodiment of the present invention.

In another embodiment of the bacterial application mode of the present invention, the bacteria do cause disease and naturally contain a gene encoding a hypersensitive response elicitor polypeptide or protein. Examples of such bacteria are noted above. However, in this embodiment, these bacteria are applied to plants or their seeds which are not susceptible to the disease carried by the bacteria. For example, Erwinia amylovora causes disease in apple or pear but not in tomato. However, such bacteria will elicit a hypersensitive response in tomato. Accordingly, in accordance with this embodiment of the present invention, Erwinia amylovora can be applied to tomato plants or seeds to enhance growth without causing disease in that species.

Another aspect of the present invention is a method which is carried out by providing a plant cell, transforming the plant cell with (i) a first DNA molecule encoding a transcript or a protein or polypeptide which confers a trait to a plant grown from the transformed plant cell and (ii) a second DNA molecule encoding a hypersensitive response elicitor protein or polypeptide which is different than the protein or polypeptide encoded by the first DNA molecule, the transforming being carried out under conditions effective to produce a transformed plant cell, and then regenerating a transgenic plant from the transformed plant cell. By transforming the plant cell with the second DNA molecule encoding a hypersensitive response elicitor protein or polypeptide, as discussed infra, the resulting transgenic plant expresses the hypersensitive response elicitor and exhibits enhanced growth, stress tolerance, disease resistance, or insect resistance.

According to one embodiment, transforming with the second DNA molecule imparts enhanced growth, stress tolerance, disease resistance, or insect resistance to the plant, thereby maximizing benefit to the plant of the trait conferred by transforming with the first DNA molecule. For example, when the particular trait conferred by the first DNA molecule relates to specific but limited growth enhancement, stress tolerance, disease resistance, or insect resistance of a transgenic plant, this embodiment relates to conferring broad growth enhancement, stress tolerance, disease resistance, or insect resistance that complements the specific but limited trait.

According to another embodiment, transforming with the first DNA molecule is accompanied by a deleterious effect on growth, stress tolerance, disease resistance, or insect resistance, and transforming with the second DNA molecule overcomes the deleterious effect. Thus, this aspect of the present invention is also directed to overcoming a yield penalty resulting from a trait.

Any of the above-described DNA molecules encoding a hypersensitive response elicitor protein or polypeptide can be used to prepare a desired transgenic plant that expresses both a transgene conferring a value-added trait and a transgene encoding a hypersensitive response elicitor.

The transgene or DNA molecule conferring a trait can be any DNA molecule that confers a value-added trait to a transgenic plant. The value-added trait can be for disease resistance, insect resistance, enhanced growth, herbicide resistance, stress tolerance, male sterility, modified flower color, or biochemically modified plant product. Biochemically modified plant products can include, without limitation, modified cellulose in cotton, modified ripening of fruits or vegetables, modified flavor of fruits or vegetables, modified flower color, expression of industrial enzymes, modified starch content, modified dietary fiber content, modified sugar metabolism, modified food quality or nutrient content, and bioremediation.

The transgene or DNA molecule conferring a value-added trait can encode either a transcript (sense or antisense) or a protein or polypeptide which is different from the hypersensitive response elicitor protein or polypeptide. Either the transcript or the protein or polypeptide, or both, can confer the value-added trait.

A number of proteins or polypeptides which can confer a value-added trait are known in the art and others are continually being identified, isolated, and expressed in host plants. Suitable proteins or polypeptides which can be encoded by the transgene or DNA molecule conferring a value-added trait include, without limitation, B.t. toxin, Photorhabdus luminescens protein, protease inhibitors, amylase inhibitors, lectins, chitinases, endochitinase, chitobiase, defensins, osmotins, crystal proteins, virus proteins, herbicide resistance proteins, mannitol dehydrogenase, PG inhibitors, ACC degradation proteins, barnase, phytase, fructans, invertase, and SAMase.

A number of transcripts which can confer a value-added trait are known in the art and others are continually being identified, isolated, and expressed in host plants. The transcript encoded by the transgene or DNA molecule conferring a trait can be either a sense RNA molecule, which is translatable or untranslatable, or an antisense RNA molecule capable of hybridizing to a target RNA or protein. Suitable transcripts which can be encoded by the transgene or DNA molecule conferring a trait include, without limitation, translatable and untranslatable RNA transcripts capable of interfering with plant virus pathogenesis (de Haan et al., “Characterization of RNA-Mediated Resistance to Tomato Spotted Wilt Virus in Transgenic Tobacco Plants,” BioTechnology 10:1133-1137 (1992); Pang et al., “Nontarget DNA Sequences Reduce the Transgene Length Necessary for RNA-Mediated Tospovirus Resistance in Transgenic Plants,” Proc. Natl. Acad. Sci. USA94:8261-8266 (1997), which are hereby incorporated by reference in their entirety) and antisense RNA molecules which interfere with the activity of an enzyme (e.g., starch synthase, ACC oxidase, pectinmethylesterase, polygalacturonase, etc.) or the synthesis of a particular product (e.g., glycoalkaloid synthesis).

Exemplary expression products of the transgene or DNA molecule conferring a trait and their uses are identified in Table 1 below.

TABLE 1
Expression Products of Transgene Conferring Value-Added
Trait and Their Uses

| Trait and Expression Product | Reference |
|---|---|
| Pest/Pathogen Resistance | |
| B.t. toxin | U.S. Pat. No. 5,990,383 to Warren et al. |
| crystal proteins | U.S. Pat. No. 4,996,155 to Sick et al. |
| Photohabdus luminescens | Bowen et al., Science 280:2129 (1998) |
| protein | |
| protease inhibitors | Ryan, Annu. Rev. Phytopathol. 38:425-449 |
| | (1990) |
| amylase inhibitors | Mundy et al., Planta 169:51-63 (1986) |
| lectins | EP Patent Application No. 351,924 A to Shell |
| chitinase (nematode & fungal) | U.S. Pat. No. 5,290,687 to Suslow et al. |
| endochitinase & chitobiase | U.S. Pat. No. 5,378,821 to Harman et al. |
| endochitnase activity | U.S. Pat. No. 5,446,138 to Blaiseu et al |
| defensins | U.S. Pat. No. 4,705,777 to Lehrer et al. |
| osmotins | Liu et al., PNAS USA 91:1888 (1994) |
| tobacco mosaic virus | Beachy et al., Rev. Phytopathol. 28:451-474 |
| coat protein | (1990) |
| cucumber mosaic virus coat protein | U.S. Pat. No. 5,349,128 to Quemada et al. |
| potato coat protein | U.S. Pat. No. 4,970,168 to Tumer et al. |
| potato leaf roll virus coat protein | U.S. Pat. No. 5,304,730 to Lawson et al. |
| potato virus replicase | U.S. Pat. No. 5,503,999 to Jilka et al. |
| | U.S. Pat. No. 5,510,253 to Mitsky et al. |
| potyvirus coat protein | WO 90/02184 to Gonsalves et al. |
| Herbicide Resistance | |
| glyphosate resistance | U.S. Pat. No. 4,535,060 to Comai et al. |
| (EPSP synthase protein) | |
| chiorsulfuron resistance | Haughn et al., Mol. Gen. Genet. 211:266 (1988) |
| phosphinothriun/bialaphos resistance | De Block, EMBO J. 6:2513 (1987) |
| Improved Nutrient Content | |
| protein | U.S. Pat. No. 6,057,493 to Willmitzer et al. |
| vitamins | U.S. Pat. No. 5,750,872 to Bennett et al. |
| oils | Shintani et al., Plant Physiol. 114(3):881-886 |
| | (1997); |
| | U.S. Pat. No. 6,069,298 to Gengenbach et al. |
| Stress Tolerance | |
| cold | U.S. Pat. No. 5,891,859 to Thomashow et al. |
| metals | U.S. Pat. No. 5,668,294 to Meaghar et al. |
| drought | U.S. Pat. No. 5,563,324 to Tarczynski et al. |
| | U.S. Pat. No. 5,780,709 to Adams et al. |
| Secondary Compounds | |
| PHB | Poirier et al., Science 256:520 (1992); |
| | Poirier et al., Bio/Technology 13:142 (1995) |
| antibodies | Tavladorki et al., Nature 366:469 (1993) |
| pharmaceutical peptides | EP Patent Application No. 436,003 A to |
| | Sijmons et al. |
| Improved Fiber | |
| cotton | U.S. Pat. No. 5,932,713 to Kasukabe et al. |
| Modified Ripening | |
| PG inhibition | U.S. Pat. No. 5,942,657 to Bird et al. |
| block ethylene synthesis: ACC | U.S. Pat. No. 5,723,766 to Theologis et al.; |
| degradation | U.S. Pat. No. 5,886,164 to Bird et al. |
| 5-adenosylmethionine hydrolase | U.S. Pat. No. 5,723,746 to Bestwick et al. |
| Male Sterility | |
| bamase | Hartley, J. Mol. Biol. 202:913 (1988) |
| (Bacillus amyloliquefaciens) | |
| ribonucleases | EP Patent No. 344,029 to Mariani et al. |
| (RiNase T1 from Asperqillus oryzae) | |
| Industrial Enzymes | |
| phytase | U.S. Pat. No. 5,593,963 to Van Ooijen et al.; |
| | Van Hartingsveldt et al., Gene 127:87 (1993) |
| Flower Color | |
| pH gene products | U.S. Pat. No. 5,534,660 to Chuck et al. |
| | U.S. Pat. No. 5,910,627 to Chuck et al. |
| dihydroflavonol 4-reductase | U.S. Pat. No. 5,410,096 to Meyer et al. |
| flavonoid biosynthetic pathway gene | U.S. Pat. No. 5,034,323 to Jorgensen et al. |
| Starch Content | |
| anti-sense starch synthase | U.S. Pat. No. 6,057,493 to Willmitzer et al. |
| amylose content | U.S. Pat. No. 6,066,782 to Kossman et al. |
| Dietary Fiber | |
| potato increased fructans | U.S. Pat. No. 5,986,173 to Smeekens et al. |
| Improved Flavor | |
| alcohol dehydrogenase II | U.S. Pat. No. 6,011,199 to Speirs et al. |
| pH gene products | U.S. Pat. No. 5,534,660 to Chuck et al. |
| | U.S. Pat. No. 5,910,627 to Chuck et al. |
| sweetness (monellin/thaumatin) | U.S. Pat. No. 5,739,409 to Fischer et al. |
| Bioremediation | |
| metalothionein in Brassicaceae | U.S. Pat. No. 5,364,451 to Raskin et al. |
| Modified Sugar Metabolism | |
| invertase | U.S. Pat. No. 5,917,127 to Willmitzer et al. |
| Modified Food Quality | |
| altered carbohydrate composition | WO 90/12876 to Gausing et al. |
| increased glutenin (wheat & others) | U.S. Pat. No. 5,914,450 to Blechi et al. |
| increased storage lipids in seed | U.S. Pat. No. 5,914,449 to Murase et al. |

Each of the references listed in Table 1 is hereby incorporated by reference in its entirety.

To express, in plant tissues, the DNA molecule encoding a hypersensitive response elicitor protein or polypeptide and/or the DNA molecule conferring a value-added trait, the coding regions must be ligated to appropriate regulatory regions which are operable in plant tissues. Therefore, plant expressible promoters and 3′ polyadenylation regions must be ligated to the DNA molecules to afford a transgene which can then be used to transform plant cells or tissues.

Any plant-expressible promoter can be utilized regardless of its origin, i.e., viral, bacterial, plant, etc. Without limitation, two suitable promoters include the nopaline synthase promoter (Fraley et al., “Expression of Bacterial Genes in Plant Cells,” Proc. Natl. Acad. Sci. USA 80:4803-4807 (1983), which is hereby incorporated by reference in its entirety) and the cauliflower mosaic virus 35S promoter (O'Dell et al., “Identification of DNA Sequences Required for Activity of the Cauliflower Mosaic Virus 35S Promoter,” Nature, 313(6005):810-812 (1985), which is hereby incorporated by reference in its entirety). Both of these promoters yield constitutive expression of coding sequences under their regulatory control.

While constitutive expression is generally suitable for expression of transgenes, it should be apparent to those of skill in the art that temporally or tissue regulated expression may also be desirable, in which case any regulated promoter can be selected to achieve the desired expression. Typically, the temporally or tissue regulated promoters will be used in connection with DNA molecules that are expressed at only certain stages of development or only in certain tissues.

For example, the E4 and E8 promoters of tomato have been used to direct fruit-specific expression of a DNA sequence in transgenic tomato plants (Cordes et al., Plant Cell 1:1025-1034 (1989); Deikman et al., EMBO J. 7:3315-3320 (1988); and Della Penna et al., Proc. Natl. Acad. Sci. USA 83:6420-6424 (1986), which are hereby incorporated by reference in their entirety). Another fruit-specific promoter is the PG promoter (Bird et al., Plant Molec. Biol. 11:651-662 (1988), which is hereby incorporated by reference). Another tissue-specific promoter is the AP2 promoter from the ovule-specific BEL1 gene promoter described in Reiser et al., Cell 83:735-742 (1995), which is hereby incorporated by reference in its entirety.

Promoters useful for expression in seed tissues include, without limitation, the promoters from genes encoding seed storage proteins, such as napin, cruciferin, phaseolin, and the like (see U.S. Pat. No. 5,420,034 to Kridl et al., which is hereby incorporated by reference in its entirety). Other suitable promoters include those from genes encoding embryonic storage proteins.

Promoters useful for expression in leaf tissue include the Rubisco small subunit promoter.

Promoters useful for expression in tubers, particularly potato tubers, include the patatin promoter.

In another embodiment of the present invention, expression of one or both transgenes is environmentally-regulated, i.e., through the use of an inducible promoter. Examples of environmental conditions that may affect transcription by inducible promoters include anaerobic conditions, elevated temperature, or the presence of light. In some plants, it may also be desirable to use promoters which are responsive to pathogen infiltration or stress. For example, it may be desirable to limit expression of the hypersensitive response elicitor protein or polypeptide in response to infection by a particular pathogen of the plant. One example of a pathogen-inducible promoter is the gstl promoter from potato, which is described in U.S. Pat. Nos. 5,750,874 and 5,723,760 to Strittmayer et al., which are hereby incorporated by reference in their entirety.

Expression of the transgenes in isolated plant cells or tissue or whole plants also requires appropriate transcription termination and polyadenylation of mRNA. Any 3′ regulatory region suitable for use in plant cells or tissue can be operably linked to the coding regions in the transgenes. A number of 3′ regulatory regions are known to be operable in plants. Exemplary 3′ regulatory regions include, without limitation, the nopaline synthase 3′ regulatory region (Fraley, et al., “Expression of Bacterial Genes in Plant Cells,” Proc. Nat'l. Acad. Sci. USA, 80:4803-4807 (1983), which is hereby incorporated by reference in its entirety) and the cauliflower mosaic virus 3′ regulatory region (Odell, et al., “Identification of DNA Sequences Required for Activity of the Cauliflower Mosaic Virus 35S Promoter,” Nature, 313(6005):810-812 (1985), which is hereby incorporated by reference in its entirety).

The promoter and a 3′ regulatory region can readily be ligated to DNA molecules using well known molecular cloning techniques described in Sambrook et al., Molecular Cloning: A Laboratory Manual, Second Edition, Cold Spring Harbor Press, NY (1989), which is hereby incorporated by reference in its entirety.

In some instances, it may be desirable for the hypersensitive response elicitor to be secreted by the cells in which it is expressed into intercellular regions of the plant. Thus, it may be desirable to ligate a DNA molecule encoding a secretion signal to the coding region of the transgene coding for the hypersensitive response elicitor protein or polypeptide. A number of suitable secretion signals are known in the art and others are continually being identified. The secretion signal can be an RNA leader which directs secretion of the subsequently transcribed protein or polypeptide, or the secretion signal can be an amino terminal peptide sequence that is recognized by a host plant secretory pathway. Typically, the DNA molecule encoding the secretion signal can be ligated between the promoter and the coding region using known molecular cloning techniques as indicated above.

An exemplary secretion signal is the secretion signal polypeptide for PRl-b gene of Nicotiana tabacum. The DNA molecule encoding this secretion signal has a nucleotide sequence corresponding to SEQ. ID. No. 15 as follows:

| | |
|---|---|
| cacgaagctt accatgggat tttttctctt ttcacaaatg ccctcatttt ttcttgtgtc | 60 |
| gacacttctc ttattcctaa taatatctca ctcttctcat gcccaaaact cccgcggrga | 120 |

The polypeptide encoded by this nucleic acid molecule has an amino acid sequence corresponding to SEQ. ID. No. 16 as follows:

```
Met Gly Phe Phe Leu Phe Ser Gln Met Pro Ser Phe Phe Leu Val Ser
    1 5 10 15
Thr Leu Leu Leu Phe Leu Ile Ile Ser His Ser Ser His Ala Gln Asn
                          20 25 30
Ser Arg Gly
                  35
```

Once transgenes of the type described above have been prepared, they can be introduced into plant cells or tissues for subsequent regeneration of whole plants. Thus, another aspect of the present invention relates to a transgenic plant which has been treated or genetically modified so that the transgenic plant can either exhibit enhanced growth, disease resistance, stress resistance, or insect resistance to realize the maximum benefit of a value-added trait or otherwise overcome a yield penalty concomitant with a value-added trait.

According to a one embodiment, the transgenic plant of the present invention includes a DNA molecule encoding a transcript or a protein or polypeptide that confers a trait, wherein the transgenic plant or a plant seed from which the transgenic plant is grown, is treated with a hypersensitive response elicitor protein or polypeptide under conditions effective to impart enhanced growth, disease resistance, stress resistance, or insect resistance to the transgenic plant.

According to another embodiment, the transgenic plant of the present invention including a first DNA molecule encoding a transcript or a protein or polypeptide that confers a trait and a second DNA molecule encoding a hypersensitive response elicitor protein or polypeptide different than the protein or polypeptide encoded by the first DNA molecule. Because the transgenic plant includes at least two DNA molecules, the first and second DNA molecules can be inserted into a plant cell or tissue either individually (i.e., in separate constructs used during separate transformation steps) or simultaneously (i.e., in a single construct or in separate constructs used during a single transformation step).

Another aspect of the present invention relates to a system for use in transforming plants with multiple DNA molecules, typically although not exclusively during separate transformation events. This system includes a first DNA construct that includes a DNA molecule encoding a transcript or a protein or polypeptide which confers a trait to a host plant, and a second DNA construct that contains a DNA molecule encoding a hypersensitive response elicitor protein or polypeptide which is different from the protein or polypeptide encoded by the DNA molecule of the first DNA construct. The first and second DNA molecules can be of the type described above. The first and second DNA constructs each contain a promoter operably linked 5′ to the DNA molecule (e.g., first or second DNA molecule) and a 3′ regulatory region operably linked to the DNA molecule.

A further aspect of the present invention relates to a DNA construct for use in transforming plants with multiple DNA molecules, typically during a single transformation event. The DNA construct includes a first DNA molecule encoding a transcript or a protein or polypeptide which confers a value-added trait to a host plant and a second DNA molecule encoding a hypersensitive response elicitor protein or polypeptide which is different from any protein or polypeptide encoded by the first DNA molecule. The first and second DNA molecules can be of the type described above. The DNA construct can include a first promoter operable in plant cells operably linked 5′ to one or both of the first and second DNA molecules. Alternatively, where the first promoter is only operably linked to the first DNA molecule, the DNA construct can also include a second promoter operably coupled to the second DNA molecule. The first and second promoters can be the same or different. Generally, both the first and second DNA molecules will be ligated to a 3′ regulatory region, which can be the same or different for each of the first and second DNA molecules.

Both the transgene or DNA molecule conferring a value-added trait and the transgene or DNA molecule encoding the hypersensitive response elicitor protein or polypeptide can be incorporated into cells using conventional recombinant DNA technology. Generally, this involves inserting the transgenes or DNA molecules into expression vector(s) or system(s) to which they are heterologous (i.e., not normally present). Because either single or multiple expression systems can be used, a single expression system can include a vector into which is inserted both the first DNA construct containing the first DNA molecule and the second DNA construct containing the second DNA molecule. Alternatively, the expression system can include two vectors into which are inserted one or the other of the first DNA construct containing the first DNA molecule and the second DNA construct containing the second DNA molecule. The first and second DNA molecules can be ligated to the appropriate promoter(s) and 3′ regulatory regions either before insertion into the expression vector(s) or system(s) or at the time of their insertion therein.

U.S. Pat. No. 4,237,224 issued to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms, typically bacteria, and eukaryotic cells grown in tissue culture, typically plant cells.

As indicated above, several aspects of the present invention are directed to the preparation of transgenic plants. Basically, this is carried out by providing a plant cell (which may or may not already possesses a transgene), transforming the plant cell with one or more transgenes of the type described above under conditions effective to yield expression of such transgenes, and then regenerating the transformed cells into whole transgenic plants. Preferably the transgene(s) is stably inserted into the genome of the transformed plant cell and whole plants regenerated therefrom.

One approach to transforming plant cells with the transgenes or DNA molecules identified herein is particle bombardment (also known as biolistic transformation) of the host cell. This can be accomplished in one of several ways. The first involves propelling inert or biologically active particles at cells. This technique is disclosed in U.S. Pat. Nos. 4,945,050, 5,036,006, and 5,100,792, all to Sanford, et al., which are hereby incorporated by reference in their entirety. Generally, this procedure involves propelling inert or biologically active particles at the cells under conditions effective to penetrate the outer surface of the cell and to be incorporated within the interior thereof. When inert particles are utilized, the vector can be introduced into the cell by coating the particles with the vector(s) containing the DNA to be used in transforming the plant cell. Alternatively, the target cell can be surrounded by the vector(s) so that the vector(s) is carried into the cell by the wake of the particle. Biologically active particles (e.g., dried bacterial cells containing the vector and DNA) can also be propelled into plant cells. Other variations of particle bombardment, now known or hereafter developed, can also be used.

Another method of introducing the transgenes or DNA molecules identified herein is fusion of protoplasts with other entities, either minicells, cells, lysosomes, or other fusible lipid-surfaced bodies that contain the first and second transgenes or DNA molecules. Fraley et al., Proc. Natl. Acad. Sci. USA, 79:1859-63 (1982), which is hereby incorporated by reference in its entirety.

The transgenes or DNA molecules identified herein may also be introduced into the plant cells by electroporation. Fromm, et al., Proc. Natl. Acad. Sci. USA, 82:5824 (1985), which is hereby incorporated by reference in its entirety. In this technique, plant protoplasts are electroporated in the presence of plasmids containing the transgenes or DNA molecules. Electrical impulses of high field strength reversibly permeabilize biomembranes allowing the introduction of the plasmids. Electroporated plant protoplasts reform the cell wall, divide, and regenerate.

Another method of introducing the transgenes or DNA molecules identified herein into plant cells is to infect a plant cell with Agrobacterium tumefaciens or Agrobacterium rhizogenes previously transformed with one or both of the transgenes or DNA molecules identified herein. Under appropriate conditions known in the art, the transformed plant cells are grown to form shoots or roots, and develop further into plants. Generally, this procedure involves inoculating the plant tissue with a suspension of bacteria and incubating the tissue for 48 to 72 hours on regeneration medium without antibiotics at 25-28° C.

Agrobacterium is a representative genus of the Gram-negative family Rhizobiaceae. Its species are responsible for crown gall (A. tumefaciens) and hairy root disease (A. rhizogenes). The plant cells in crown gall tumors and hairy roots are induced to produce amino acid derivatives known as opines, which are catabolized only by the bacteria. The bacterial genes responsible for expression of opines are a convenient source of control elements for chimeric expression cassettes. In addition, assaying for the presence of opines can be used to identify transformed tissue.

The transgenes or DNA molecules identified herein can be introduced into appropriate plant cells by means of the Ti plasmid of A. tumefaciens or the Ri plasmid of A. rhizogenes. The Ti or Ri plasmid is transmitted to plant cells upon infection by Agrobacterium and is stably integrated into the plant genome. Schell, J., Science, 237:1176-83 (1987), which is hereby incorporated by reference in its entirety.

Plant tissue suitable for transformation include leaf tissue, root tissue, meristems, zygotic and somatic embryos, and anthers.

After transformation, the transformed plant cells can be selected and regenerated.

Preferably, transformed cells are first identified using, e.g., a selection marker simultaneously introduced into the host cells along with the transgene or DNA molecules identified herein. Suitable selection markers include, without limitation, markers coding for antibiotic resistance, such as kanamycin resistance (Fraley, et al., Proc. Natl. Acad. Sci. USA, 80:4803-4807 (1983), which is hereby incorporated by reference in its entirety). A number of antibiotic-resistance markers are known in the art and other are continually being identified. Any known antibiotic-resistance marker can be used to transform and select transformed host cells in accordance with the present invention. Cells or tissues are grown on a selection media containing an antibiotic, whereby generally only those transformants expressing the antibiotic resistance marker continue to grow.

Once a recombinant plant cell or tissue has been obtained, it is possible to regenerate a transgenic plant of the present invention. Plant regeneration from cultured protoplasts is described in Evans et al., Handbook of Plant Cell Cultures, Vol. 1: (MacMillan Publishing Co., New York, 1983); and Vasil I. R. (ed.), Cell Culture and Somatic Cell Genetics of Plants, Acad. Press, Orlando, Vol. I, 1984, and Vol. III (1986), which are hereby incorporated by reference in their entirety.

It is known that practically all plants can be regenerated from cultured cells or tissues, including but not limited to, all major crop and medicinal plant species, trees, perennial and annual ornamental plants, and turf and ornamental grasses. Exemplary crop species include, without limitation, rice, wheat, barley, rye, cotton, sunflower, peanut, corn, potato, sweet potato, bean, pea, chicory, lettuce, endive, cabbage, canola, cauliflower, broccoli, turnip, radish, spinach, onion, garlic, eggplant, pepper, celery, carrot, squash, pumpkin, zucchini, cucumber, apple, pear, melon, strawberry, cranberry, grape, raspberry, pineapple, soybean, tobacco, tomato, sorghum, and sugarcane. Exemplary trees include, without limitation, maple, birch, oak, walnut, cherry, pine, and poplar. Exemplary ornamental plants include, without limitation, begonias, impatiens, geraniums, lilies, daylilies, irises, tulips, and roses.

Means for regeneration vary from species to species of plants, but generally a suspension of transformed protoplasts or a petri plate containing transformed explants is first provided. Callus tissue is formed and shoots may be induced from callus and subsequently rooted. Alternatively, embryo formation can be induced in the callus tissue. These embryos germinate as natural embryos to form plants. The culture media will generally contain various amino acids and hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

After the transgenes or DNA molecules identified herein are stably incorporated in transgenic plants, they can be transferred to other plants by sexual crossing or by preparing cultivars. With respect to sexual crossing, any of a number of standard breeding techniques can be used depending upon the species to be crossed. Cultivars can be propagated in accord with common agricultural procedures known to those in the field. Once transgenic plants of this type are produced, the plants themselves can be cultivated in accordance with conventional procedures. Alternatively, transgenic seeds or propagules (e.g., scion or rootstock cultivars) are recovered from the transgenic plants.

A further aspect of the present invention relates to a method of making a transgenic plant which includes providing a transgenic plant seed containing both the transgene or DNA molecule conferring the trait and the transgene or DNA molecule encoding the hypersensitive response elicitor protein or polypeptide, and then planting the transgenic seed under conditions effective to grow a transgenic plant from the transgenic seed. Although any medium can be used to germinate and grow the transgenic seeds, preferably they are planted in the soil and cultivated using conventional procedures to produce the transgenic plants. Preferably, the transgenic plant seed is harvested from a transgenic parent plant as described above. Thus, the transgenic plants are propagated from the planted transgenic seeds under conditions effective to confer the value-added trait and hypersensitive response elicitor protein or polypeptide expression to subsequent generations.

Another method for preparing a transgenic plant of the present invention involves providing two distinct transgenic plant lines, one containing the transgene or DNA molecule conferring the trait stably inserted into its genome and the other containing the transgene or DNA molecule encoding the hypersensitive response elicitor protein or polypeptide stably inserted into its genome. The two lines are then crossed using conventional breeding techniques and the resulting generation segregated and self-crossed to propagate a single hybrid line which possesses the value-added trait conferred by expression of the first transgene or DNA molecule and expresses the hypersensitive response elicitor protein or polypeptide encoded by the second transgene or DNA molecule. Additional value-added traits can be crossed into such a transgenic hybrid line.

### EXAMPLES

The following examples are provided to illustrate embodiments of the present invention, but they are by no means intended to limit its scope.

### Example 1

### Increased Yields from Transgenic Cotton Varieties Treated with Messenger

Field trials designed to test the effects of Messenger® on disease resistance and crop yield in cotton were performed using the following transgenic cotton varieties: Delta and Pine Land (“DPL”) 20B, DPL 33B, DPL 35B, DPL 50B, Stoneville BXN 47, and Paymaster 1220BR. All of the DPL cotton varieties are transgenic for genes encoding Bt toxin, which confers resistance to a specific class of insects. Stoneville BXN 47 is a transgenic cotton variety with a gene for resistance to the herbicide bromoxynil. Paymaster 1220BR is a transgenic variety with stacked transgenic traits: in addition to the Bt toxin gene, this variety carries a second gene conferring resistance to the herbicide glyphosate. The transgenic traits in all six varieties have specific functions limited to providing insect resistance and/or resistance to herbicide. The transgenes are not intended to alter capacity for growth and yield so the characteristics of the non-transgenic parental varieties are retained in the transgenic varieties.

In the eight field trials where the cotton varieties were transgenic, Messenger® was applied by foliar spray or combined seed treatment and foliar spray. In five of these trials, different numbers of treatments and rates of application were tested. Yields were measured in lbs lint/acre or in lbs seed cotton/acre. The results of the trials are summarized in Table 2 below.

TABLE 2
Increased Yields From Messenger ® Treated Transgenic Cotton

| | Cotton | | | Percent |
|---|---|---|---|---|
| Trial | Variety | Treatment | Rate | Yield Increase |
| 1 | DPL 20B | 4 foliar | 2.2 oz./acre | 11.4 |
| 2 | DPL 33B | 3 foliar | 2.2 oz./acre | 11.4 |
| 3 | DPL 33B | 3 foliar | 2.2 oz./acre | 5.9 |
| | | 3 foliar | 4.4 oz./acre | 9.5 |
| 4 | DPL 50B | 3 foliar | 2.29 oz./acre | 1.0 |
| | | 3 foliar | 4.59 oz./acre | 15.0 |
| 5 | DPL 35B | 3 foliar | 2.2 oz./acre | 6.0 |
| | | seed and | 2.2 oz./50 lb. | |
| | | 3 foliar | 2.2 oz./acre | 26.0 |
| | | seed and | 2.2 oz./50 lb. | |
| | | 3 foliar | 4.4 oz./acre | 33.0 |
| 6 | Paymaster 1220BR | 3 foliar | 2.2 oz./acre | 4.7 |
| 7 | Paymaster 1220BR | 3 foliar | 2.2 oz./acre | 13.1 |
| | | 4 foliar | 2.2 oz./acre | 11.8 |
| | | seed and | 2 oz./cwt | |
| | | 3 foliar | 2.2 oz./acre | 11.5 |
| | | seed and | 2 oz./cwt | |
| | | 3 foliar | 4.4 oz./acre | 16.2 |
| 8 | Stoneville BXN 47 | 3 foliar | 2.2 oz./acre | 49.6 |
| | | 4 foliar | 2.2 oz./acre | 60.4 |
| | | seed and | 2 oz./cwt | 14.0 |
| | | 3 foliar | 2.2 oz./acre | |
| | | seed and | 2 oz./cwt | 38.8 |
| | | 3 foliar | 4.4 oz./acre | |

DPL varieties 20B (trial 1) and 33B (trial 2), and Paymaster 1220BR (trial 6) were treated with spray applications of Messenger® at 2.2 oz./acre starting at the first true leaf stage, followed by early bloom and mid bloom applications. Yields from treated plants were higher than for the untreated control plants of the same varieties with increases ranging from 4.7% to 11.4%. Messenger® was applied at rates of 2.2 and 4.4 oz./acre for a second trial on DPL 33B (trial 3) and at rates of 2.29 and 4.59 oz/acre on DPL 50B (trial 4). Spray applications for these two trials were at first true leaf, early bloom, and mid bloom. In both trials, the high rate applications gave higher yields than the lower rate applications. The dose/response effect of Messenger® on yield is strong evidence that the observed yield increases were a direct result of the Messenger® applications.

In trials on DPL 35B (trial 5), Stoneville BXN 47 (trial 8), and a second trial with Paymaster 1220BR (trial 7), multiple types of treatments were carried out including foliar spray and foliar spray combined with seed treatment, with varying numbers of applications and application rates. Foliar applications were made at rates of 2.2 oz./acre or 4.4 oz./acre and from three to four times during the season. Multiple foliar applications were also made in combination with seed treatment at 2 oz./cwt or 2.2 oz./50lbs. Foliar Messenger® applications were made beginning at first true leaf, followed by early bloom and mid bloom applications. All treatments gave increased yields over untreated control plants of the same varieties. In each of these three trials, low and high rate applications were made and effects on increased yield showed a dose response correspondence with the amount of Messenger® applied.

The average increase in yield for all applications in all eight trials was 19.6%. For those trials with low and high rate applications, the average yield increases were 14.6% for low rate applications and 25.8% for the high rate applications.

### Example 2

### Increased Fruit Number in Transgenic Cotton Varieties Treated with Messenger®

Cotton yields can be directly impacted by the total number of bolls produced per plant. In three of the eight trials presented in Example 1 (trials 2, 6, and 7), analysis of the effects of Messenger® treatment on yield were extended to include a comparison of the numbers of bolls produced by treated and untreated transgenic cotton. The results are summarized in Table 3 below.

TABLE 3
Increased Fruit Number in Messenger ® Treated Transgenic Cotton

| | | | | Plant Mapping 1 | | Plant Mapping 2 | |
|---|---|---|---|---|---|---|---|
| | Cotton | | | Fruit per | Percent | Fruit per | Percent |
| Trial | Variety | Treatment | Rate | Plant | Increase | Plant | Increase |
| 2 | DPL 33B | control | — | 8.73 | — | 10.2 | — |
| | | 3 foliar | 2.2 oz./acre | 10.78 | 23.5 | 11.0 | 7.8 |
| 6 | Paymaster | | | | | | |
| | 1220BR | control | — | 38.1 | — | 14.8 | — |
| | | 3 foliar | 2.2 oz./acre | 42.1 | 10.4 | 17.6 | 18.9 |
| 7 | Paymaster | | | | | | |
| | 1220BR | control | — | 10.5 | — | | |
| | | 3 foliar | 2.2oz./acre | 11.1 | 5.7 | − | — |
| | | 4 foliar | 2.2 oz./acre | 12.6 | 20.0 | − | — |
| | | seed and | 2 oz./cwt | 11.6 | 10.5 | − | — |
| | | 3 foliar | 2.2 oz./acre | | | | |
| | | seed and | 2 oz./cwt | 11.3 | 7.9 | — | − |
| | | 3 foliar | 4.4 oz./acre | | | | |

In trials performed with DPL 33B (trial 2) and Paymaster 1220BR (trial 6), plants received three spray treatments with Messenger®. Applications were made as described in Example 1. Two boll counts were made in each trial, once after early bloom and a second time near harvest. A higher number of bolls was present on treated plants in both trial 2 and 6, at each of the early and late season plant mappings ranging from 7.8% to 23.5% increase in number over control plants.

A second trial performed with Paymaster 1220BR (trial 7) was carried out with four types of treatments, as indicated in Table 3. A late season plant mapping revealed increased boll numbers for all Messenger® treated plants compared with untreated Paymaster 1220BR plants, with increases ranging from 5.7% to 20.0%.

The six Messenger® treatments in the two trials resulted in higher yields than obtained from untreated control plants. The results of these trials indicate that an increase in boll number can be a contributing factor to increased yields obtained from Messenger® treated cotton. There is an important to distinction to be made between effects on yield from Messenger® and effects on yield resulting from transgenic traits conferred by insect or herbicide resistance genes such as those in the transgenic cotton varieties in these trials. Such resistance genes do not increase the basic yield characteristics of the transgenic plant but simply reduce yield losses caused by insect or weed pressure. A combination of Messenger® and such resistance genes would have complementary effects on yield since Messenger® would provide a higher baseline yield through its effects on growth such as increased fruit number, while resistance genes such as Bt toxin would act to preserve that higher yield by reducing losses to insect pressure.

### Example 3

### Increased Number of Open Bolls on Transgenic Cotton Treated with Messenger

The number of open bolls present at harvest is a factor in total yield. A trial including four different types of Messenger® treatments on the transgenic cotton variety Stoneville BXN 47 gave higher yields than obtained from untreated Stoneville BXN 47 (trial 8, Table 2). In addition to the measurements of overall yields, observations were extended to include a comparison of the numbers of open bolls at harvest on the Messenger® treated plants and untreated control plants. Four types of Messenger® treatments were tested in this trial. Two treatments consisted of either three or four foliar applications at rates of 2.2 oz./acre. The remaining two treatments consisted of seed application combined with foliar sprays using 2 oz/cwt for seed treatments and 2.2 or 4.4 oz./acre for three foliar applications. Open bolls were counted at three positions on the plants. Position 1 corresponded to the lowest node with bolls. Position 2 corresponded to the next node above on the stem. Position 3 included bolls at the third node and above combined into a single total. The totals for numbers of bolls at all three positions were also calculated. The results of this analysis are summarized in Table 4 below.

TABLE 4
Increased Number of Open Boils From Messenger ® Treated Transgenic Cotton

| | | | | Open Boils | | | | |
|---|---|---|---|---|---|---|---|---|
| | Cotton | | | Position | | | Per | Percent |
| Trial | Variety | Treatment | Rate | 1 | 2 | 3 | Plant | Increase |
| 8 | Stoneville | control | — | 3.56 | 0.33 | 0.00 | 3.89 | — |
| | BXN 47 | | | | | | | |
| | | 3 foliar | 2.2 oz./acre | 5.11 | 1.78 | 0.00 | 6.89 | 77.1 |
| | | 4 foliar | 2.2 oz./acre | 5.45 | 1.45 | 0.11 | 7.00 | 79.9 |
| | | seed and | 2 oz./cwt | | | | | |
| | | 3 foliar | 2.2 oz./acre | 544 | 1.22 | 0.00 | 6.67 | 71.5 |
| | | seed and | 2 oz./cwt | | | | | |
| | | 3 foliar | 4.4 oz./acre | 5.22 | 2.00 | 0.11 | 7.33 | 88.4 |

The four types of Messenger® treatments performed in the trial resulted in increased numbers of open bolls on Stoneville BXN 47 relative to untreated Stoneville BXN 47. Increases in open bolls ranged from 71.5% to 88.4%. A dose response effect of Messenger® treatment on open boll number was evidenced by a higher percentage increase in open bolls with applications made at a rate of 4.4 oz./acre compared to applications made at 2.2 oz./acre.

### Example 4

### Increased Yield from Transgenic Cotton Grown in a Field Infested with Reniform Nematodes

Nematodes are parasitic worms that live in the soil and attack the roots of cotton. In an infested field, reniform nematodes can cause a 10-25% loss in yield and as much as 50% loss under stress conditions such as drought. A field trial to test effects of Messenger® treatment on cotton under nematode pressure was conducted in a field known to be infested with reniform nematodes. The cotton variety in the trial was Stoneville BXN 47, identified in Example 1. Since the bromoxynil transgene cannot provide resistance to nematodes, this cotton variety is just as susceptible to damage by nematodes as non-transgenic varieties.

Messenger® treatments of four types were applied in this trial. Two treatments consisted of either three or four foliar applications at rates of 2.2 oz./acre. The two other treatments consisted of seed application using 2 oz./cwt combined with three foliar sprays at rates of either 2.2 or 4.4 oz./acre. Foliar applications were made at first true leaf followed by early and mid bloom applications. Yields from treated and untreated plots of Stoneville BXN 47 were determined as well as nematode populations in the soil from the plots. Results are summarized in Table 5 below.

TABLE 5
Increased Yield From Messenger ® Treated Transgenic
Cotton Grown in Nematode Infested Field

| | | | | Nematode Population | | | |
|---|---|---|---|---|---|---|---|
| | Cotton | | | At | At | Percent | Yield |
| Trial | Variety | Treatment | Rate | Planting | Harvest | Change | Increase |
| 8 | Stoneville | control | — | 9927 | 7609 | −23.4 | − |
| | BXN 47 | | | | | | |
| | | 3 foliar | 2.2 oz./acre | 8889 | 6953 | −21.8 | 49.6 |
| | | 4 foliar | 2.2 oz./acre | 8807 | 5948 | −32.5 | 60.4 |
| | | seed and | 2 oz./cwt | | | | |
| | | 3 foliar | 2.2 oz./acre | 6528 | 4867 | −25.4 | 14.0 |
| | | seed and | 2 oz./cwt | | | | |
| | | 3 foliar | 4.4 oz./acre | 10622 | 7957 | −25.1 | 38.8 |

Yields were substantially higher in all four plots receiving Messenger® treatment compared to untreated plots. The increased yields in response to Messenger® could be due to enhanced growth effects, induced resistance to nematodes, or a combination of both. Nematode populations declined over the course of the growing season with no significant difference in the amount of decline between treated and untreated plots, indicating that Messenger® did not directly affect nematodes. The significantly lower yield from the untreated Stoneville BXN 47 plot demonstrates the reserve potential for higher yield in a transgenic variety that can be elicited by Messenger®.

### Example 5

### Application of Messenger® to Bt-transformed Corn Changes Toxicity Profile to Fall Armyworm

Non-Bt-transformed corn, (Yellow-sugary, 83-d maturity, cv. “Rogers”, F1 Bonus, from Novartis) and Bt-transformed corn, (cv. “Rogers”, GH-0937, also from Novartis) were planted in pots (one plant per pot, four replicate pots) and then placed in a greenhouse under normal conditions. When plants were 2-3 feet tall (pre-tassel), they were treated with a single foliar spray of Messenger® at a rate of 3 oz/acre in approximately 40 gal/acre. The concentration of harpin_{Ea} (active ingredient) in this spray was approximately 17 ppm.

Five days after the application of Messenger®, leaf discs of approximately 0.5 inch in diameter were collected from treated and non-treated plants and placed in on agar media in petri dishes. Fall armyworm (FAW, Spotoptera frugiperda) neonate larvae were added to each petri dish. Leaf discs were replaced as needed in order to provide a constant food supply to the larvae.

At 6 days after treatment (DAT), feeding activity by FAW was measured by counting the number of leaf disks completely eaten in both transformed and non-transformed corn, treated with and without Messenger®. As demonstrated in Table 6 below, substantial feeding activity occurred in both Messenger® and non-Messenger® treated, non-transformed corn. However, in Bt-transformed corn, very little feeding activity occurred.

TABLE 6
Feeding Activity and Mortality Data for
Fall Armyworm Feeding on Messenger ®
Treated and Non-treated Bt-corn and non-Bt-corn

| | | Feeding* | Mortality | |
|---|---|---|---|---|
| Corn Description | Treatment | 6 DAT | 7 DAT | 8 DAT |
| Non-transformed | — | 27 | 0% | 0% |
| Non-transformed | Messenger ® | 34 | 0% | 0% |
| Bt-transformed | — | 2 | 30% | 30% |
| Bt-transformed | Messenger ® | 0 | 80% | 80% |

*Number of leaf disks completely eaten by 20 larvae, with “0” indicating no leaf disks entirely eaten.
DAT = days after treatment.

At 7 and 8 DAT no larval mortality was recorded in non-Bt-transformed corn, whether treated with Messenger® or not. However, in Bt-transformed corn, mortality at both 7 and 8 DAT was substantially lower for Messenger®-treated compared to non-Messenger®-treated (Table 6).

The increased mortality of FAW in Messenger®-treated, Bt-transformed corn suggests that application of Messenger® may have synergistic effects at controlling larval feeding activity.

### Example 6

### Herbicide Resistant Transgenic Crops

A variety of technologies have been developed for production of transgenic plants resistant to herbicides including glyphosate, Synchrony, glufosinate, sethoxydim, imidazolinone, bromoxynil, and sulfonylurea. Each of these technologies relies on the introduction of a single gene that confers resistance to a particular herbicide. Since the introduced gene is limited to a single function, other agronomically important traits of the crop plants remain unmodified. Glyphosate resistant transgenic cotton, soybean, and canola, for example, are susceptible to the same range of diseases that affect the non-transgenic parental lines from which the transgenic lines were developed. Yield losses due to disease could be minimized by combining genes for herbicide resistance and hypersensitive response elicitor expression in the same transgenic plant, thereby allowing the full benefits of the herbicide resistance trait to be realized.

### Example 7

### Insect Resistant Transgenic Crops

Bt toxin protein from the soil bacterium Bacillus thuringiensis has been used on crops for many years as a topically applied insecticide with activity against specific classes of insects. A large number of genes have been isolated that encode different versions of the Bt toxin protein with varying specificities in insecticidal activity. The introduction of a gene encoding Bt toxin into potato was one of the first commercial applications of transgenic technology in crop plants. Since then, the commercialization of insect resistant crops expressing Bt toxin genes has been extended to include cotton and corn, with other crops under development. The specific insect resistance function of the Bt toxin gene is generally effective, but disease resistance and growth traits remain unaltered in the transgenic crops expressing Bt toxin genes. While yield losses due to insect pressure are reduced in Bt toxin expressing crops, they are still vulnerable to losses caused by pathogens. Bringing Bt toxin genes together with a transgene coding for hypersensitive response elicitor expression would produce crops that are resistant to pathogens as well as insects. An additional benefit would be increased yield due to the enhanced growth effect of the hypersensitive response elicitor.

### Example 8

### Transgenic Crops with Enhanced Nutrient Value

Transgenic technology can be used to modify the balance of nutrients in crops to eliminate nutritional deficiencies. Some food crops are naturally deficient in particular amino acids that are a necessary component of the human diet. Cereal crops are often poor in tryptophan and lysine while vegetable crops and legume crops such as soybean are low in cysteine and methionine. Amino acids present at low amounts can be increased to nutritionally useful levels through the introduction of a gene encoding a protein with a high content of a particular amino acid that is normally lacking. Another approach that allows improvement of nutritional value is modification of an existing biochemical pathway or introduction of a novel biochemical pathway by introduction of a transgene. This can result in production of a compound with nutritional value that is normally absent or present in low amounts. Rice is an important food crop worldwide but is naturally low in vitamin A. Transgenic rice with increased vitamin A content could help to alleviate dietary deficiencies in this nutrient and is currently being developed. Transgenes can also be used to modify fatty acid biosynthesis pathways so as to produce food oils with altered levels of saturation. This method of improving nutritional value has been applied to canola, soybean, and flax so far. An aspect common to all the above approaches for enhanced nutritional quality is that improvements to the crops are limited to nutritional characteristics. Disease resistance and overall growth and yield properties of the crops remain unimproved. Combining a transgene coding for hypersensitive response elicitor expression with genes that confer enhanced nutritional value would allow the generation of transgenic crops that maximize the nutritional advantages through reduced losses to diseases and through improved yields due to enhanced growth.

### Example 9

### Compensation for Transgenic Trait-Associated Losses in Yield

Introduction of a transgene for a beneficial trait can on occasion result in the introduction of a disadvantageous quality. For example, evidence indicates that the glyphosate resistance trait by itself can result in reduced yield in crops expressing the resistance gene. A study done at the University of Wisconsin compared 1998 yields from glyphosate resistant soybean crops with yields from non-transgenic varieties at multiple sites in 8 Midwestern states and New York. At a majority of sites, yields of the glyphosate resistant soybeans were significantly lower than the non-transgenic varieties. The growth enhancement effect of a hypersensitive response elicitor could act to decrease or eliminate the yield penalty if combined with herbicide resistance genes in transgenic plants.

Introduction of a transgene may on occasion result in the loss of an advantageous trait. New Mexico State University reported losses to fungal infection in transgenic cotton varieties during the 1998 cotton season. Paymaster varieties that were insect resistant due to the presence of a Bt toxin transgene were susceptible to Verticillium wilt. Since the non-transgenic varieties had been resistant to Verticillium wilt, the introduction of the Bt toxin gene had resulted in loss of the fungal resistance trait. Negative side effects on disease resistance that might result from introduction of a transgene could be reduced or eliminated by combination with a transgene coding for hypersensitive response elicitor expression, which actively confers a broad range of disease resistance.

### Example 10

### Pathogen Resistant Transgenic Crops

Crops are subject to attack by viral, bacterial, and fungal pathogens. An extensive amount research has been devoted to identifying ways to make crops resistant to pathogen attack. As a result, a growing number of genes have been identified that confer or have potential to confer pathogen resistance when expressed in transgenic plants. A major limitation of the resistance genes characterized so far is they have restricted ranges of effectiveness. A gene may confer resistance to viral but not fungal or bacterial pathogens, and vice versa. In many cases the protection is more narrowly limited to a small subset of viral, bacterial, or fungal pathogens. Transgenic plants expressing any of these resistance genes have reduced susceptibility to attack by specific pathogens or classes of pathogens, but the narrow range of resistance leaves the plants vulnerable to attack by many other pathogens. An example of how a narrow range of protection conferred by a transgene can leave a crop vulnerable to non-target organisms was demonstrated by substantial losses in Bt toxin cotton in Texas in 1996 to non-target pests. Hypersensitive response elicitor expression is effective in providing resistance against many viral, bacterial, and fungal pathogens. Combining the transgene coding for a hypersensitive response elicitor with resistance genes that are narrowly focused in transgenic plants would provide a broader range of protection and decreased losses.

Although the invention has been described in detail for the purposes of illustration, it is understood that such detail is solely for that purpose, and variations can be made therein by those skilled in the art without departing from the spirit and scope of the invention which is defined by the following claims.

What is claimed:

1. A method comprising:
providing a plant or plant seed comprising a transgene conferring a transgenic trait to the plant or a plant grown from the plant seed, and
applying to the plant or plant seed a hypersensitive response elicitor protein or polypeptide.

2. The method according to claim 1, wherein said applying is carried out under conditions effective to impart enhanced growth, stress tolerance, disease resistance, or insect resistance to the plant or the plant grown from the plant seed, thereby maximizing the benefit of the transgenic trait to the plant or the plant grown from the plant seed.

3. The method according to claim 2, said applying is carried out on a plant.

4. The method according to claim 3, wherein said applying is carried out by spraying, injection, dusting, or leaf abrasion at a time proximate to when said applying takes place.

5. The method according to claim 2, wherein said applying is carried out on a plant seed.

6. The method according to claim 5, wherein said applying is carried out by spraying, injection, coating, dusting, or immersion.

7. The method according to claim 2, wherein the hypersensitive response elicitor polypeptide or protein is applied to the plant or plant seed as a composition further comprising a carrier.

8. The method according to claim 7, wherein the carrier is selected from the group consisting of water, aqueous solutions, slurries, and powders.

9. The method according to claim 7, wherein the composition contains greater than 0.5 nM of the hypersensitive response elicitor polypeptide or protein.

10. The method according to claim 2, wherein the hypersensitive response elicitor polypeptide or protein is in isolated form.

11. The method according to claim 2, wherein the hypersensitive response elicitor protein or polypeptide is derived from a species of pathogen selected from the group consisting of Erwinia, Xanthomonas, Pseudomonas, Phytophthora, and Clavibacter.

12. The method according to claim 1, wherein the transgenic trait is associated with a deleterious effect on growth, stress tolerance, disease resistance, or insect resistance in the transgenic plant and said applying is carried out under conditions effective to impart enhanced growth, stress tolerance, disease resistance, or insect resistance to the plant or the plant grown from the plant seed, thereby overcoming the deleterious effect.

13. The method according to claim 12, said applying is carried out on a plant.

14. The method according to claim 13, wherein said applying is carried out by spraying, injection, dusting, or leaf abrasion at a time proximate to when said applying takes place.

15. The method according to claim 12, wherein said applying is carried out on a plant seed.

16. The method according to claim 15, wherein said applying is carried out by spraying, injection, coating, dusting, or immersion.

17. The method according to claim 12, wherein the hypersensitive response elicitor polypeptide or protein is applied to the plant or plant seed as a composition further comprising a carrier.

18. The method according to claim 17, wherein the carrier is selected from the group consisting of water, aqueous solutions, slurries, and powders.

19. The method according to claim 17, wherein the composition contains greater than 0.5 nM of the hypersensitive response elicitor polypeptide or protein.

20. The method according to claim 12, wherein the hypersensitive response elicitor polypeptide or protein is in isolated form.

21. The method according to claim 12, wherein the hypersensitive response elicitor protein or polypeptide is derived from a species of pathogen selected from the group consisting of Erwinia, Xanthomonas, Pseudomonas, Phytophthora, and Clavibacter.

22. A method comprising:
providing a plant cell;
transforming the plant cell with (i) a first DNA molecule encoding a transcript or a protein or polypeptide which confers a trait to a plant grown from the transformed plant cell and (ii) a second DNA molecule encoding a hypersensitive response elicitor protein or polypeptide which is different than the protein or polypeptide encoded by the first DNA molecule, said transforming being carried out under conditions effective to produce a transgenic plant cell; and
regenerating a transgenic plant from the transformed plant cell.

23. The method according to claim 22, wherein said transforming with the second DNA molecule imparts enhanced growth, stress tolerance, disease resistance, or insect resistance to the plant, thereby maximizing benefit to the plant of the trait conferred by said transforming with the first DNA molecule.

24. The method according to claim 22, wherein said transforming with the first DNA molecule is accompanied by a deleterious effect on growth, stress tolerance, disease resistance, or insect resistance and wherein said transforming with the second DNA molecule overcomes the deleterious effect.

25. The method according to claim 22, wherein said transforming is carried out by
transforming the plant cell with the first DNA molecule to form a singly transformed plant cell and
transforming the singly transformed plant cell with the second DNA molecule.

26. The method according to claim 22, wherein said transforming is carried out by
transforming the plant cell with the second DNA molecule to form a singly transformed plant cell and
transforming the singly transformed plant cell with the first DNA molecule.

27. The method according to claim 22, wherein said transforming is carried out by simultaneously transforming the plant cell with the first and second DNA molecules.

28. The method according to claim 22, wherein said transforming is performed under conditions effective to insert the first and second DNA molecules into the genome of the transformed plant cell.

29. The method according to claim 22, wherein said transforming is Agrobacterium mediated.

30. The method according to claim 22, wherein said transforming comprises:
propelling particles at the plant cell under conditions effective for the particles to penetrate into the cell interior and
introducing one or more expression vectors into the plant cell interior, the one or more expression vectors comprising either the first DNA molecule, the second DNA molecule, or both the first and second DNA molecules.

31. The method according to claim 22, wherein the hypersensitive response elicitor protein or polypeptide is derived from a species of pathogen selected from the group consisting of Erwinia, Xanthomonas, Pseudomonas, Phytophthora, and Clavibacter.

32. The method according to claim 22, wherein the first DNA molecule encodes a protein or polypeptide selected from the group consisting of B.t. toxin, Photorahabdus luminscens protein, protease inhibitors, amylase inhibitors, lectins, chitinases, endochitinase, chitobiase, defensins, osmotins, crystal proteins, virus proteins, and SAMase.

33. The method according to claim 22, wherein the first DNA molecule encodes a transcript selected from the group consisting of antisense RNA and sense RNA.

34. The method according to claim 22, wherein the first DNA molecule encodes antisense RNA which interferes with activity of an enzyme or synthesis of a product.

35. The method according to claim 22, wherein the first DNA molecule comprises:
a promoter operable in plants;
a DNA coding sequence operably coupled 3′ of the promoter, the DNA coding sequence encoding the transcript or the protein or polypeptide which confers the trait; and
a 3′ regulatory region operably coupled to the DNA coding sequence.

36. The method according to claim 22, wherein the second DNA molecule comprises:
a promoter operable in plants;
a DNA coding sequence operably coupled 3′ of the promoter, the DNA coding sequence encoding the hypersensitive response elicitor protein or polypeptide; and
a 3′ regulatory region operably coupled to the DNA coding sequence.

37. A transgenic plant comprising:
a first DNA molecule encoding a transcript or a protein or polypeptide that confers a trait and
a second DNA molecule encoding a hypersensitive response elicitor protein or polypeptide different than the protein or polypeptide encoded by the first DNA molecule.

38. The transgenic plant according to claim 37, wherein the first and second DNA molecules are stably inserted into the genome of the transgenic plant.

39. The transgenic plant according to claim 37, wherein the transgenic plant is selected from the group consisting of rice, wheat, barley, rye, cotton, sunflower, peanut, corn, potato, sweet potato, bean, pea, chicory, lettuce, endive, cabbage, canola, cauliflower, broccoli, turnip, radish, spinach, onion, garlic, eggplant, pepper, celery, carrot, squash, pumpkin, zucchini, cucumber, apple, pear, melon, strawberry, cranberry, grape, raspberry, pineapple, soybean, tobacco, tomato, sorghum, and sugarcane.

40. The transgenic plant according to claim 37, wherein the hypersensitive response elicitor protein or polypeptide is derived from a species of pathogen selected from the group consisting of Erwinia, Xanthomonas, Pseudomonas, Phytophthora, and Clavibacter.

41. The transgenic plant according to claim 37, wherein the trait is selected from the group consisting of disease resistance, insect resistance, enhanced growth, herbicide resistance, stress tolerance, male sterility, modified flower color, and biochemically modified plant product.

42. The transgenic plant according to claim 41, wherein the first DNA molecule encodes a protein or polypeptide selected from the group consisting of B.t. toxin, Photorahabdus luminscens protein, protease inhibitors, amylase inhibitors, lectins, chitinases, endochitinase, chitobiase, defensins, osmotins, crystal proteins, virus proteins, herbicide resistance proteins, mannitol dehydrogenase, PG inhibitors, ACC degradation proteins, barnase, phytase, fructans, invertase, and SAMase.

43. The transgenic plant according to claim 41, wherein the first DNA molecule encodes a transcript selected from the group consisting of antisense RNA and sense RNA.

44. The transgenic plant according to claim 43, wherein the first DNA molecule encodes antisense RNA which interferes with activity of an enzyme or synthesis of a product.

45. A transgenic plant seed obtained from the transgenic plant according to claim 37.

46. A system for use in transforming plants with multiple DNA molecules, said system comprising:
a first DNA construct comprising a first DNA molecule which confers a trait to a host plant, and
a second DNA construct comprising a second DNA molecule encoding a hypersensitive response elicitor protein or polypeptide.

47. The system according to claim 46, wherein the hypersensitive response elicitor protein or polypeptide is derived from a species of pathogen selected from the group consisting of Erwinia, Xanthomonas, Pseudomonas, Phytophthora, and Clavibacter.

48. The system according to claim 46, wherein the trait is selected from the group consisting of disease resistance, insect resistance, enhanced growth, herbicide resistance, stress tolerance, male sterility, modified flower color, and biochemically modified plant product.

49. The system according to claim 48, wherein the first DNA molecule encodes a protein or polypeptide selected from the group consisting of Bt toxin, Photorahabdus luminscens protein, protease inhibitors, amylase inhibitors, lectins, chitinases, endochitinase, chitobiase, defensins, osmotins, crystal proteins, virus proteins, herbicide resistance proteins, mannitol dehydrogenase, PG inhibitors, ACC degradation proteins, barnase, phytase, fructans, invertase, and SAMase.

50. The system according to claim 48, wherein the first DNA molecule encodes a transcript selected from the group consisting of antisense RNA and sense RNA.

51. The system according to claim 50, wherein the first DNA molecule encodes antisense RNA which interferes with activity of an enzyme or synthesis of a product.

52. An expression system comprising first and second vectors into which the system according to claim 46 is inserted, wherein the first DNA construct is inserted into the first vector and the second DNA construct is inserted into the second vector.

53. A transgenic host cell comprising:
a first DNA molecule encoding a transcript or a protein or polypeptide that confers a trait to a host plant and
a second DNA molecule encoding a hypersensitive response elicitor protein or polypeptide which is different than the protein or polypeptide encoded by the first DNA molecule.

54. The transgenic host cell according to claim 53, wherein the host cell is a bacterial cell or a plant cell.

55. The transgenic host cell according to claim 54, wherein the host cell is a bacterial cell.

56. The transgenic host cell according to claim 55, wherein the bacterial cell is an Agrobacterium cell.

57. The transgenic host cell according to claim 54, wherein the host cell is a plant cell.

58. The transgenic host cell according to claim 57, wherein the first and second DNA molecules are stably inserted into the genome of the plant cell.

59. The transgenic host cell according to claim 53, wherein the hypersensitive response elicitor protein or polypeptide is derived from a species of pathogen selected from the group consisting of Erwinia, Xanthomonas, Pseudomonas, Phytophthora, and Clavibacter.

60. The transgenic host cell according to claim 53, wherein the trait is selected from the group consisting of disease resistance, insect resistance, enhanced growth, herbicide resistance, stress tolerance, male sterility, modified flower color, and biochemically modified plant product.

61. The transgenic host cell according to claim 60, wherein the first DNA molecule encodes a protein or polypeptide selected from the group consisting of B.t. toxin, Photorahabdus luminscens protein, protease inhibitors, amylase inhibitors, lectins, chitinases, endochitinase, chitobiase, defensins, osmotins, crystal proteins, virus proteins, herbicide resistance proteins, mannitol dehydrogenase, PG inhibitors, ACC degradation proteins, barnase, phytase, fructans, invertase, and SAMase.

62. The transgenic host cell according to claim 60, wherein the first DNA molecule encodes a transcript selected from the group consisting of antisense RNA and sense RNA.

63. The transgenic host cell according to claim 62, wherein the first DNA molecule encodes antisense RNA which interferes with activity of an enzyme or synthesis of a product.

64. A DNA construct comprising:
a first DNA molecule which confers a trait to a host plant and
a second DNA molecule encoding a hypersensitive response elicitor protein or polypeptide.

65. The DNA construct according to claim 64, wherein the hypersensitive response elicitor protein or polypeptide is derived from a species of pathogen selected from the group consisting of Erwinia, Xanthomonas, Pseudomonas, Phytophthora, and Clavibacter.

66. The DNA construct according to claim 64, wherein the trait is selected from the group consisting of disease resistance, insect resistance, enhanced growth, herbicide resistance, stress tolerance, male sterility, modified flower color, and biochemically modified plant product.

67. The DNA construct according to claim 66, wherein first DNA molecule encodes a protein or polypeptide selected from the group consisting of B.t. toxin, Photorahabdus luminscens protein, protease inhibitors, amylase inhibitors, lectins, chitinases, endochitinase, chitobiase, defensins, osmotins, crystal proteins, virus proteins, herbicide resistance proteins, mannitol dehydrogenase, PG inhibitors, ACC degradation proteins, barnase, phytase, fructans, invertase, and SAMase.

68. The DNA construct according to claim 66, wherein the first DNA molecule encodes a transcript selected from the group consisting of antisense RNA and sense RNA.

69. The DNA construct according to claim 68, wherein the first DNA molecule encodes antisense RNA which interferes with activity of an enzyme or synthesis of a product.

70. The DNA construct according to claim 64 further comprising:
a first promoter operable in plant cells operably linked 5′ to one or both of the first and second DNA molecules and
a first 3′ regulatory region operably linked 3′ to one or both of the first and second DNA molecules.

71. The DNA construct according to claim 70, wherein the first promoter is inducible.

72. The DNA construct according to claim 70, wherein the first promoter and the first 3′ regulatory region are operably linked 5′ to the first DNA molecule but not the second DNA molecule, the DNA construct further comprising:
a second promoter operably linked 5′ to the second DNA molecule and
a second 3′ regulatory region operably linked 3′ to the second DNA molecule.

73. The DNA construct according to claim 72, wherein the first and second promoters are different.

74. An expression system comprising a vector into which is inserted a heterologous DNA construct according to claim 64.
